(19) United States
(12) Patent Application Publication
(10) Pub. No.: US 2005/0014762 A1
(43) Pub. Date: Jan. 20, 2005

(54) Combination

(76) Inventors: Rolf Beume, Konstanz (DE); Daniela Bundschuh, Konstanz (DE); Christian Weimar, Konstanz (DE); Stefan-Lutz Wollin, Meersburg (DE)

Correspondence Address: NATH & ASSOCIATES, 1030 15th STREET, NW, 6TH FLOOR, WASHINGTON, DC, 20005, US

(21) Appl. No.: 10/489,903

(22) PCT Filed: Sep. 17, 2002
(86) PCT No.: PCT/EP02/10423

(30) Foreign Application Priority Data
Sep. 19, 2001 (EP) 01000474.5

(51) Int. Cl. A61K 31/519; A61K 31/4709

(52) U.S. Cl. 514/252.16; 514/262.1; 514/314

(57) ABSTRACT

The invention relates to the combined administration of PDE4 or PDE3/4 inhibitors and leukotriene receptor antagonists for the treatment of respiratory tract disorders.

### FIELD OF APPLICATION OF THE INVENTION

The present invention relates to combinations of pharmaceutically active substances for use in the treatment of respiratory tract disorders.

The substances used in the combinations according to the invention are known active compounds from the PDE4 and PDE3/4 inhibitors class and active compounds from the class of the leukotriene receptor antagonists.

### KNOWN TECHNICAL BACKGROUND

In the International Patent Application WO02/38155 the combined use of Roflumilast, Roflumilast-N-Oxide or a pharmaceutically acceptable salt of either compound on the one hand and of a leukotriene-receptor antagonist on the other hand for the treatment of bronchial and respiratory disorders is described. In the International Patent Application WO01/90076 a pharmaceutical composition comprising Roflumilast-N-Oxide and a leukotriene receptor antagonist is claimed.

### DESCRIPTION OF THE INVENTION

Asthma is a common inflammatory disease of the respiratory tract, accounting for 1-3% of all office visits, 500,000 hospital admissions per year and more pediatric hospital admissions than any other single illness in the US. Annually, more than 5000 children and adults die of asthma attacks in the United States (William E. S.; Goodmann Gilmann A.:The pharmacological Basis of Therapeutics, 9^{th} Edition, pp. 152 & 659-682, Mc Graw Hill, New York 1996).

Asthma can no longer be viewed simply as a reversible airway obstruction. It should instead be considered primarily as an inflammatory illness that has bronchial hyperactivity and bronchospasm as its results. Allergen specific immunoglobulin E (IgE) is bound to the mast cells via Fc receptors. It is a fragment obtained by papain digestion of immunoglobulin molecules and contains most of the antigenic determinants. When an allergen comes into contact with IgE, the mast cells are activated and release a number of inflammatory mediators, which include granule contents like histamine, proteases, heparin, and tumor necrosis factor (TNF), a variety of lipid membrane derived molecules like prostaglandins, leukotrienes and platelet activating factor (PAF), and a number of cytokines like interleukin (IL)-1, 3, 4, 5, 6 and 8 and chemokines. An enormous variety of mediators are released which have more than one potent effect on airway inflammation.

As a result of vasodilation, increased vasopermeability and increased endothelial adhesiveness towards leukocytes further leads to an influx of inflammatory cells like lymphocytes, eosinophils and macrophages from blood circulation into the tissues. This in turn leads to the release of mediators which have further inflammatory effects (Rao A. R. et al. Recent Perspectives in the design of antiasthmatic agents, Pharmazie, 55, 7, 475-482, 2000).

Thus, it can be understood that it is unlikely that drugs affecting a single mediator can satisfactorily treat the disease alone. As asthma is one of the major diseases affecting mankind, there is a need to develop drugs which can affect a wide variety of mediators.

Therefore, it is the object of the present invention to make available respiratory tract therapeutics which fulfil the following conditions:
Favorable simultaneous influence on several of the inflammatory mediators
Marked bronchorelaxation and -dilatation
Good oral availability
Minor side effects
Good suitability for long-term therapy
Favorable influence on bronchial hyperreactivity

It has now been found that the combined use of a PDE4 or a PDE3/4 inhibitor and a leukotriene receptor antagonist outstandingly fulfills the above-mentioned conditions.

The invention thus relates to the combined use of a PDE4 or a PDE3/4 inhibitor and a leukotriene receptor antagonist in the treatment of respiratory tract disorders.

“Combined use” in the context of the invention means the simultaneous, sequential or separate administration of the PDE4 or the PDE3/4 inhibitor on the one hand and of the leukotriene receptor antagonist on the other hand.

Simultaneous administration includes—aside from the simultaneous uptake of two separate dosage forms containing the PDE4 or the PDE3/4 inhibitor in the one and the leukotriene receptor antagonist in the other dosage form—pharmaceutical compositions containing both active ingredients in one single dosage form (fixed unit dose form).

Sequential administration in the context of the invention means the administration of the PDE4 or the PDE3/4 inhibitor on the one hand and of the leukotriene receptor antagonist on the other hand in separate dosage forms within less than 12 hours, more preferably within less than one hour, most preferably within 5 minutes or less.

Separate administration within the context of the invention means the administration of the PDE4 or the PDE3/4 inhibitor on the one hand and of the leukotriene receptor antagonist on the other hand in separate dosage forms within 12 hours or more.

“Combined use” in the context of the invention also includes a pharmaceutical product comprising both the PDE4 or the PDE3/4 inhibitor and the leukotriene receptor antagonist as discrete separate dosage forms, in separate containers or e. g. in blisters containing both types of drugs in discrete solid dosage units, preferably in a form in which the dosage units which have to be taken together or which have to be taken within one day are grouped together in a manner which is convenient for the patient. Said pharmaceutical product itself or as a part of a kit may contain instructions for the simultaneous, sequential or separate administration of the discrete separate dosage units, to a patient in need thereof.

By the expression “PDE4 inhibitor” is meant a selective phosphodiesterase inhibitor, which inhibits preferentially the type 4 phosphodiesterase when compared to other known types of phosphodiesterase, e.g. type 1, 2, 3, 5 etc., whereby the compound has a lower IC₅₀ (more potent) for the type 4 phosphodiesterase, such as where the IC₅₀ for PDE4 inhibition is about factor 10 lower compared to IC₅₀ for inhibition of other known type of phosphodiesterase, e.g. type 1, 2, 3, 5 etc.

Analogously, the expression “PDE3/4 inhibitor” is defined. Methods to determine the activity and selectivity of a phosphodiesterase inhibitor are known to the person skilled in the art. In this connection it may be mentioned, for example, the methods described by Thompson et al. (Adv Cycl Nucl Res 10: 69-92, 1979), Giembycz et al. (Br J Pharmacol 118: 1945-1958, 1996) and the phosphodiesterase scintillation proximity assay of Amersham Pharmacia Biotech.

By the expression “leukotriene receptor antagonist” is meant leukotriene C4, leukotriene D4 and leukotriene E4 receptor antagonists, of which the leukotriene D4 receptor antagonists are preferred.

As possible PDE4 or PDE3/4 inhibitors within the meaning of the present invention may be mentioned, by way of example, those PDE4 or PDE3/4 inhibitors which are named expressis verbis as an example, or described or claimed generically in the following patent applications and patents: DE 1545687, DE 2028869, DE 2123328, DE 2315801, DE 2402908, DE 2413935, DE 3900233, EP 0103497, EP 0139464, EP 0158380, EP 0163965, EP 0335386, EP 0389282, EP 0393500, EP 0428302, EP 0435811, EP 0449216, EP 0459505, EP 0470805, EP 0490823, EP 0506194, EP 0510562, EP 0511865, EP 0527117, EP 0553174, EP 0557016, EP 0626939, EP 0664289, EP 0671389, EP 0685474, EP 0685475, EP 0685479, EP 0731099, EP 0736532, EP 0738715, EP 0748805, EP 0763534, EP 0816357, EP 0819688, EP 0819689, EP 0832886, EP 0834508, EP 0848000, JP 92234389, JP 94329652, JP 95010875, JP 98072415, JP 98147585, U.S. Pat. Nos. 5,703,098, 5,739,144, WO 9117991, WO 9200968, WO 9212961, WO 9307146, WO 9315044, WO 9315045, WO 9318024, WO 9319068, WO 9319720, WO 9319747, WO 9319749, WO 9319751, WO 9325517, WO 9402465, WO 9412461, WO 9420455, WO 9422852, WO 9427947, WO 9500516, WO 9501338, WO 9501980, WO 9503794, WO 9504045, WO 9504046, WO 9505386, WO 9508534, WO 9509623, WO 9509624, WO 9509627, WO 9509836, WO 9514667, WO 9514680, WO 9514681, WO 9517392, WO 9517399, WO 9519362, WO 9520578, WO 9522520, WO 9524381, WO 9527692, WO 9535281, WO 9535283, WO 9535284, WO 9600218, WO 9601825, WO 9606843, WO 9603399, WO 9611690, WO 9611917, WO 9612720, WO 9631486, WO 9631487, WO 9635683, WO 9636595, WO 9636596, WO 9636611, WO 9636625, WO 9636626, WO 9636638, WO 9638150, WO 9639408, WO 9640636, WO 9703967, WO 9704779, WO 9705105, WO 9708143, WO 9709345, WO 9712895, WO 9718208, WO 9719078, WO 9720833, WO 9722585, WO 9722586, WO 9723457, WO 9723460, WO 9723461, WO 9724117, WO 9724355, WO 9725312, WO 9728131, WO 9730999, WO 9731000, WO 9732853, WO 9735854, WO 9736905, WO 9740032, WO 9743288, WO 9744036, WO 9744322, WO 9747604, WO 9748697, WO 9804534, WO 9805327, WO 9806692, WO 9806704, WO 9807715, WO 9808828, WO 9808830, WO 9808841, WO 9808844, WO 9809946, WO 9809961, WO 9811113, WO 9814448, WO 9818796, WO 9821207, WO 9821208, WO 9821209, WO 9822453, WO 9831674, WO 9840382, WO 9845268, WO 9855481, WO 9856756, WO 9905111, WO 9905112, WO 9505113, WO 9906404, WO 9918095, WO 9931071, WO 9931090, WO 9947505, WO 9957115, WO 9957118, WO 9964414, WO 0001695, WO 0012501, WO 0026208, WO 0042017, WO 0042018, WO 0042019, WO 0042020, WO 0042034, WO 0119818, WO 0130766, WO 0130777, WO 0151470, WO 0206239, WO 0206270, WO 0205616 and WO 0206238.

Exemplary PDE inhibitors are shown on the following pages with the aid of their formulae:

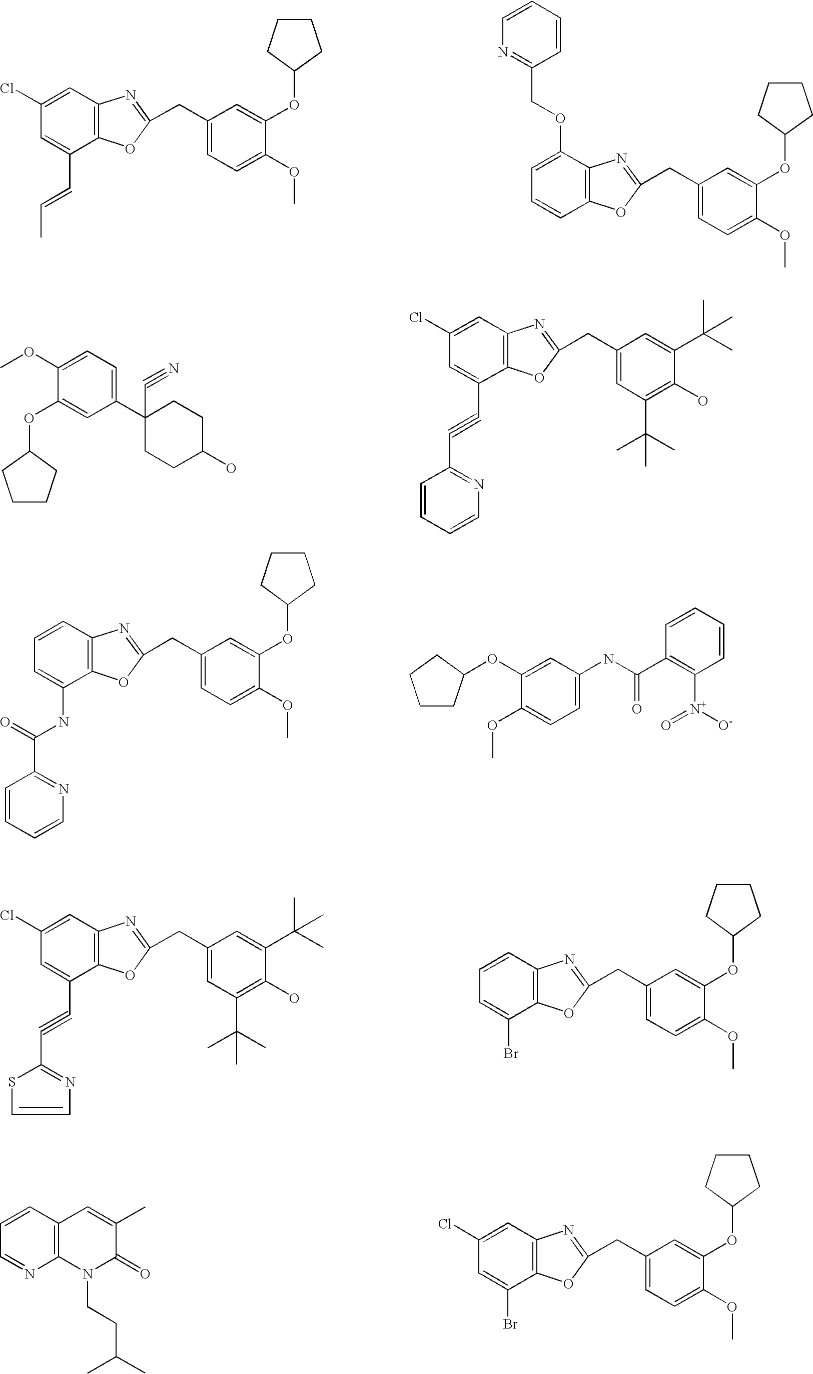

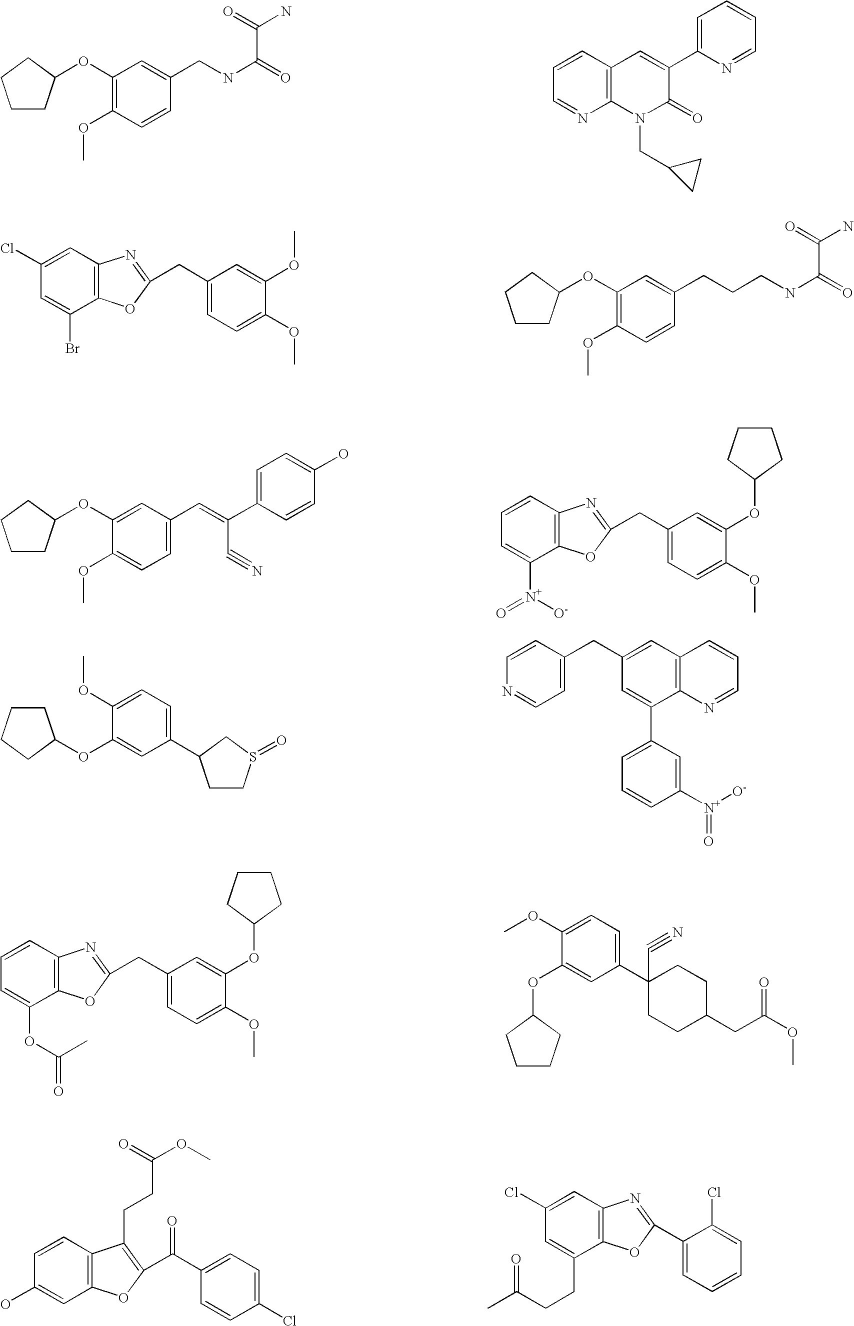

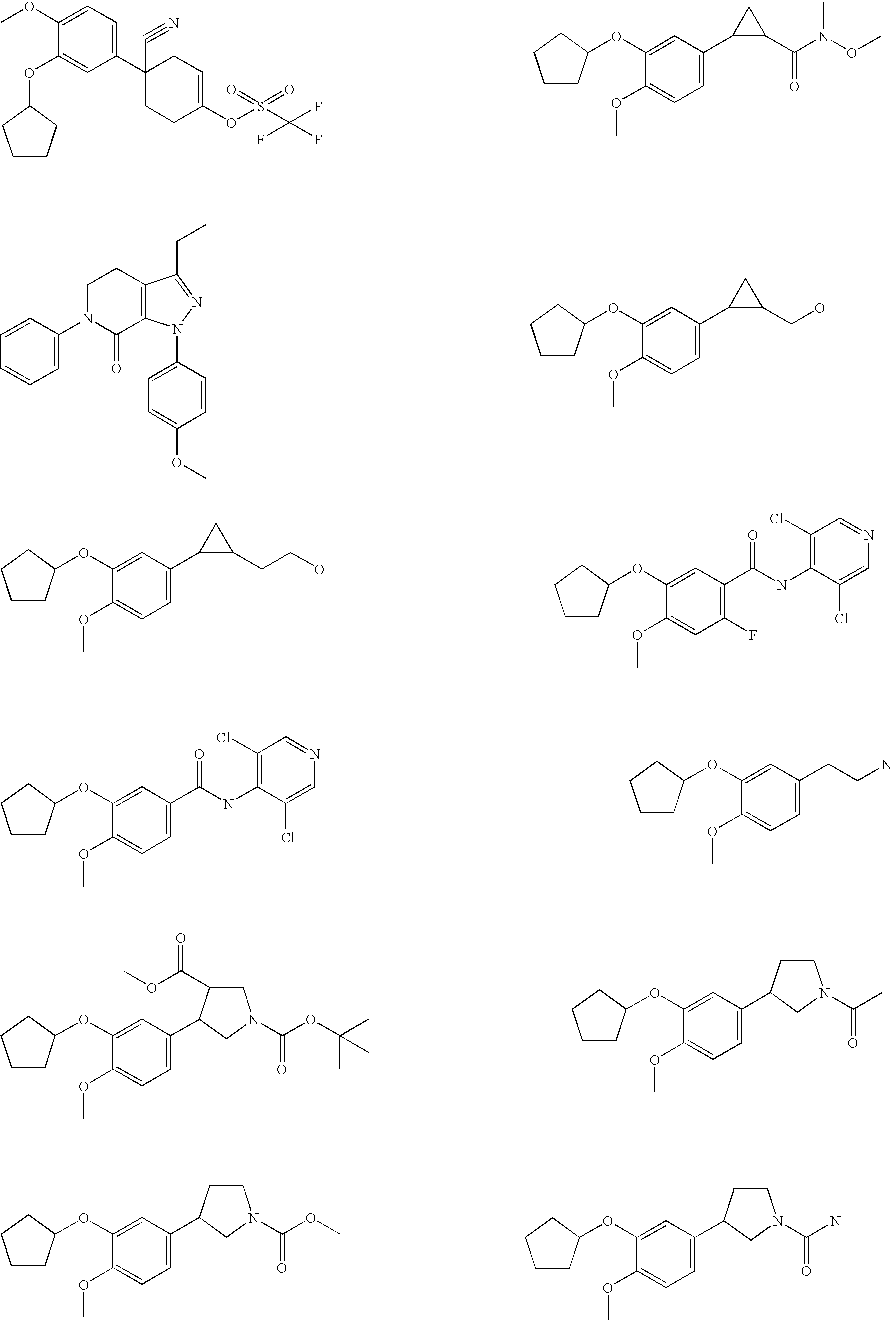

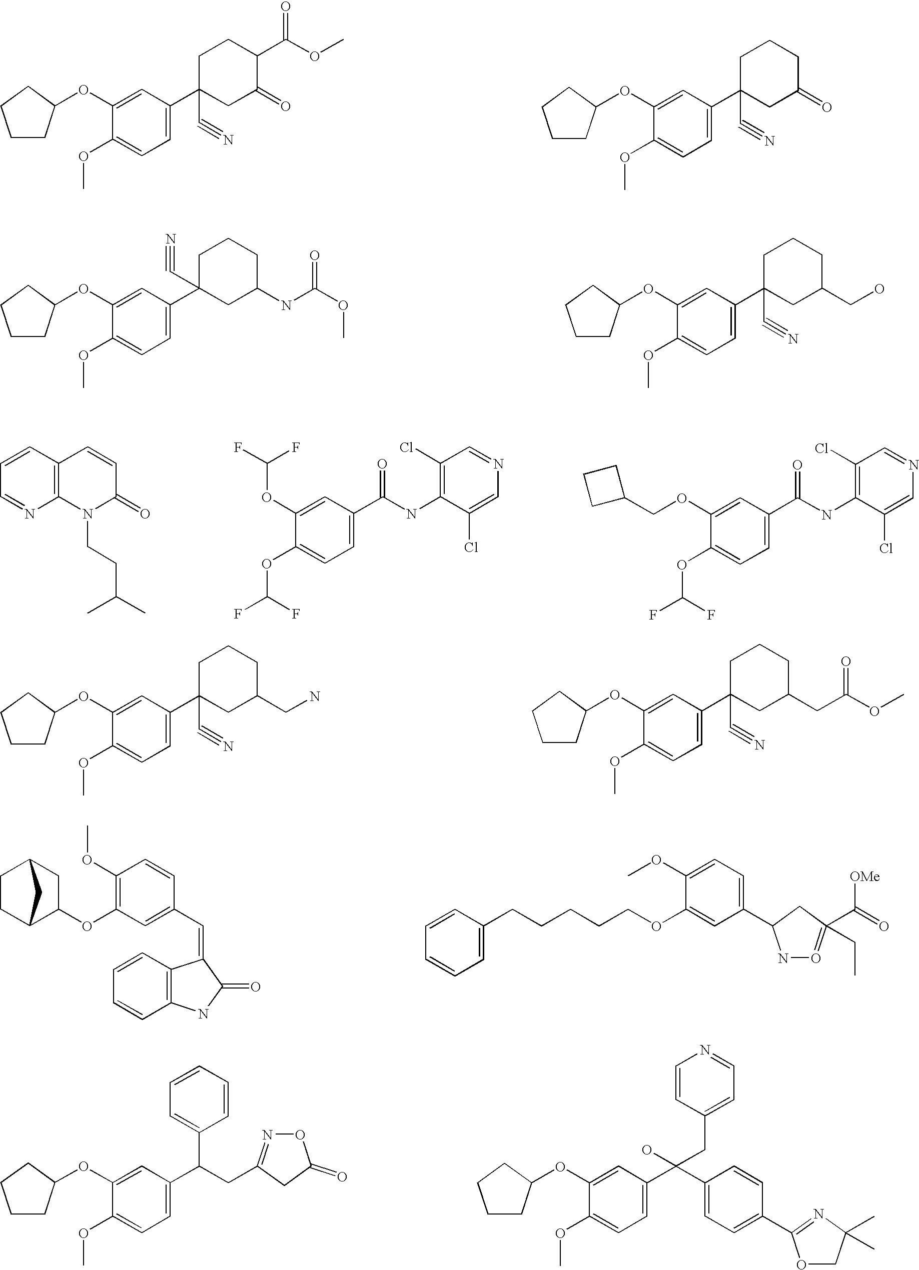

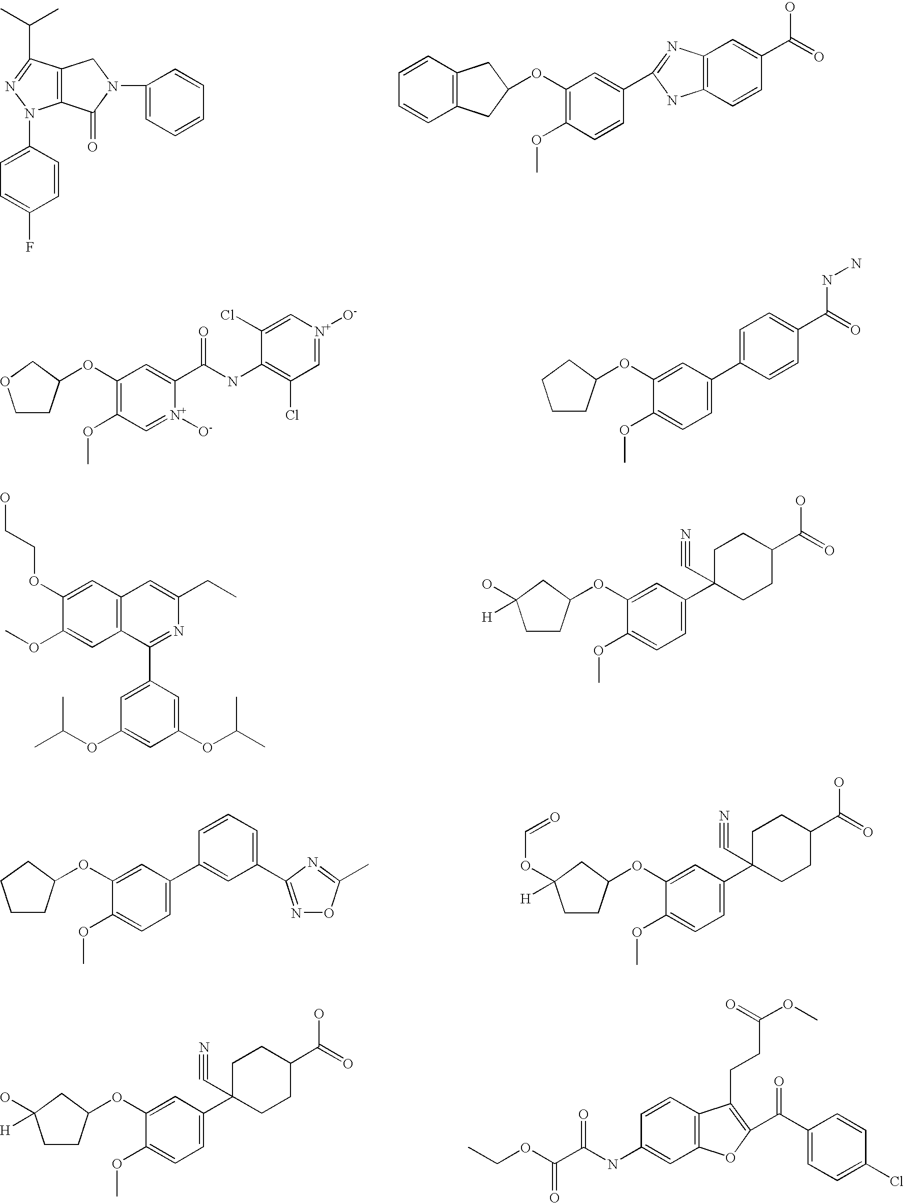

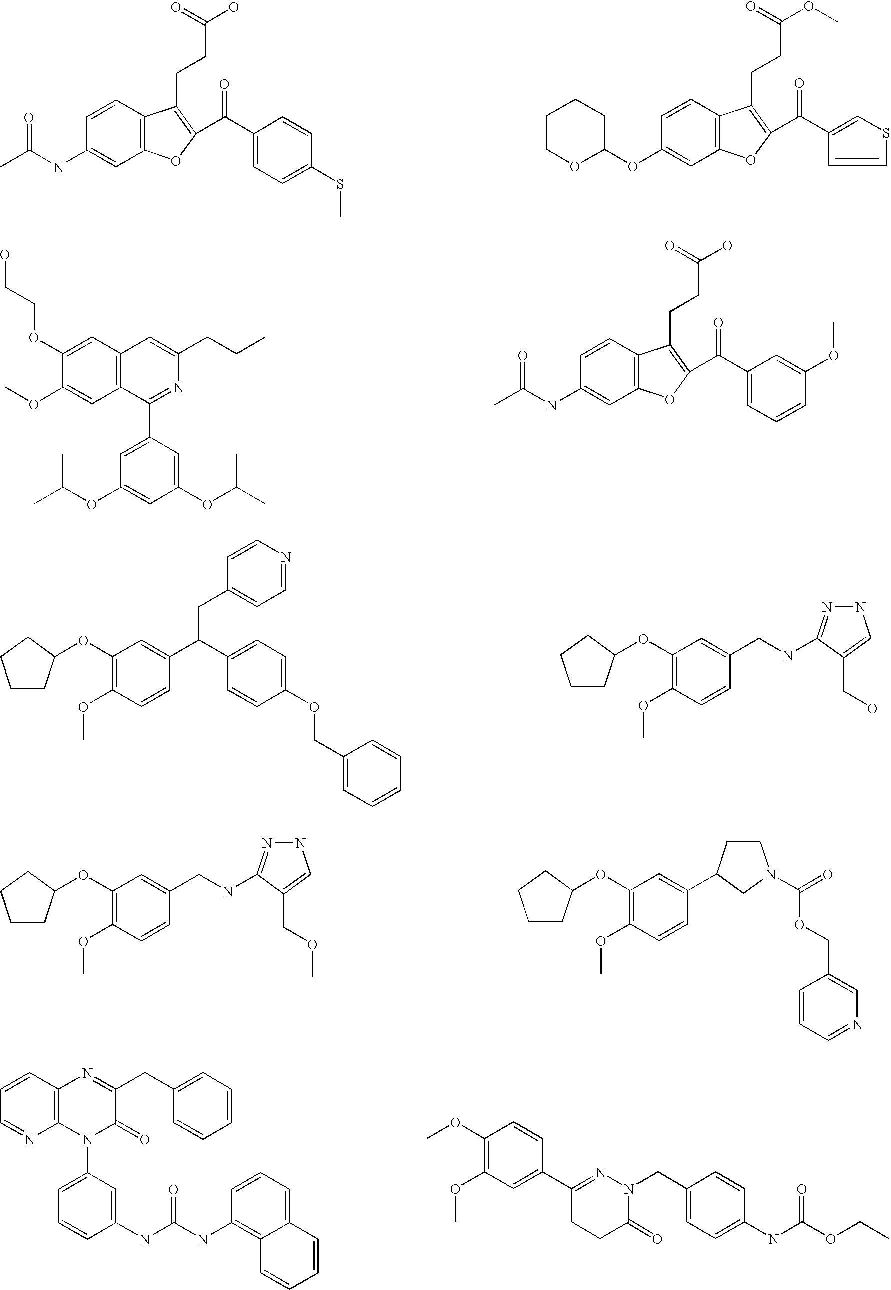

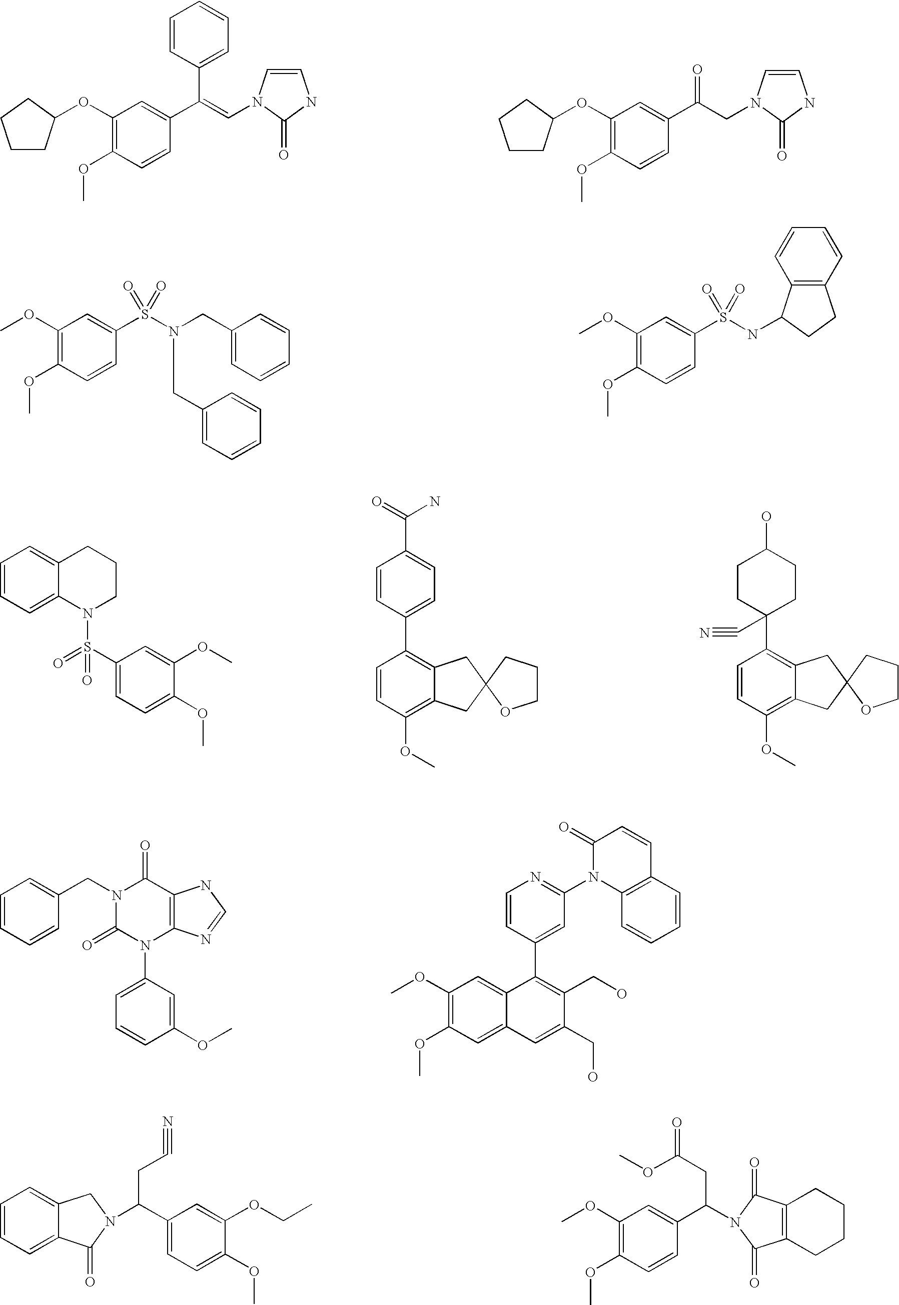

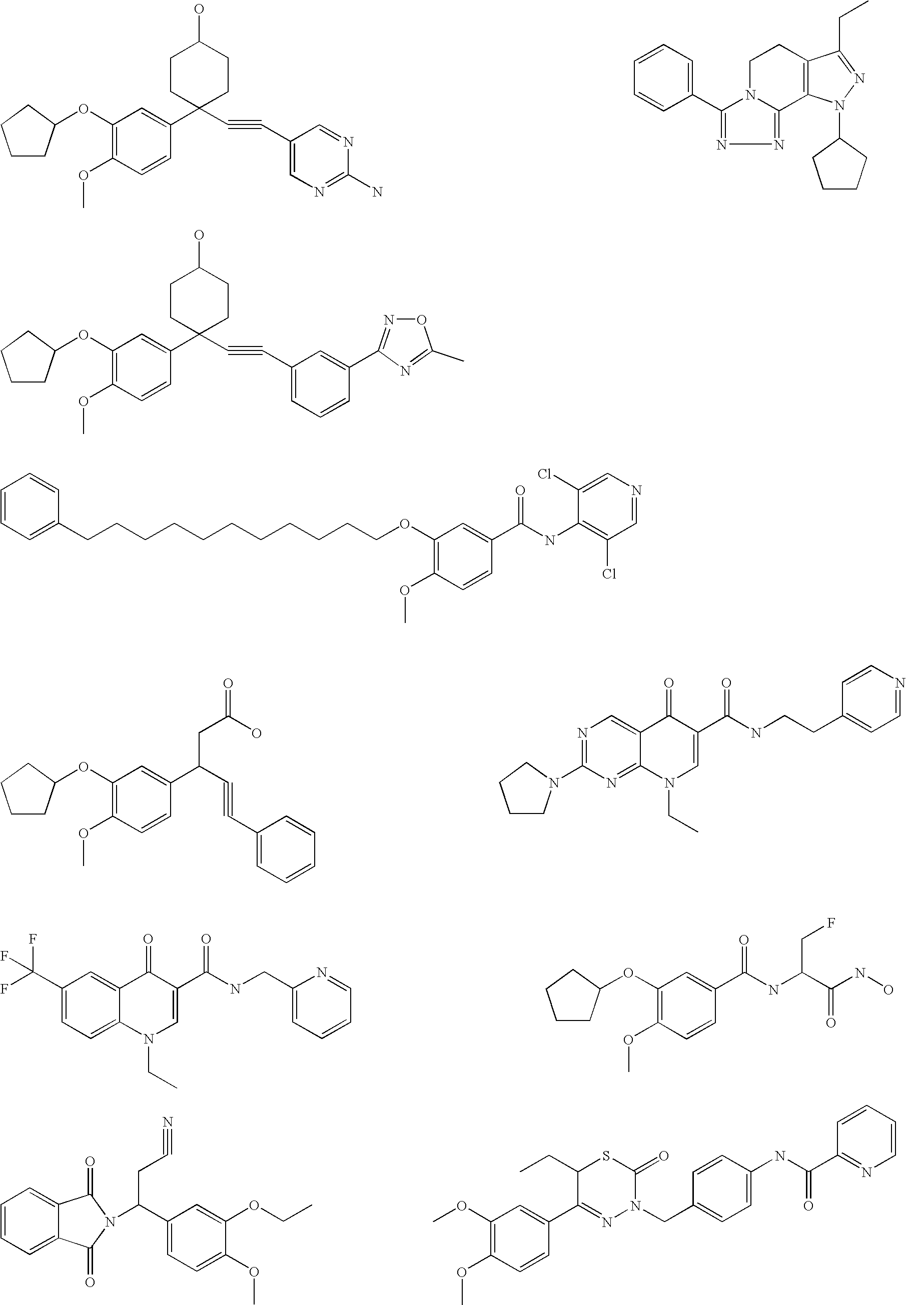

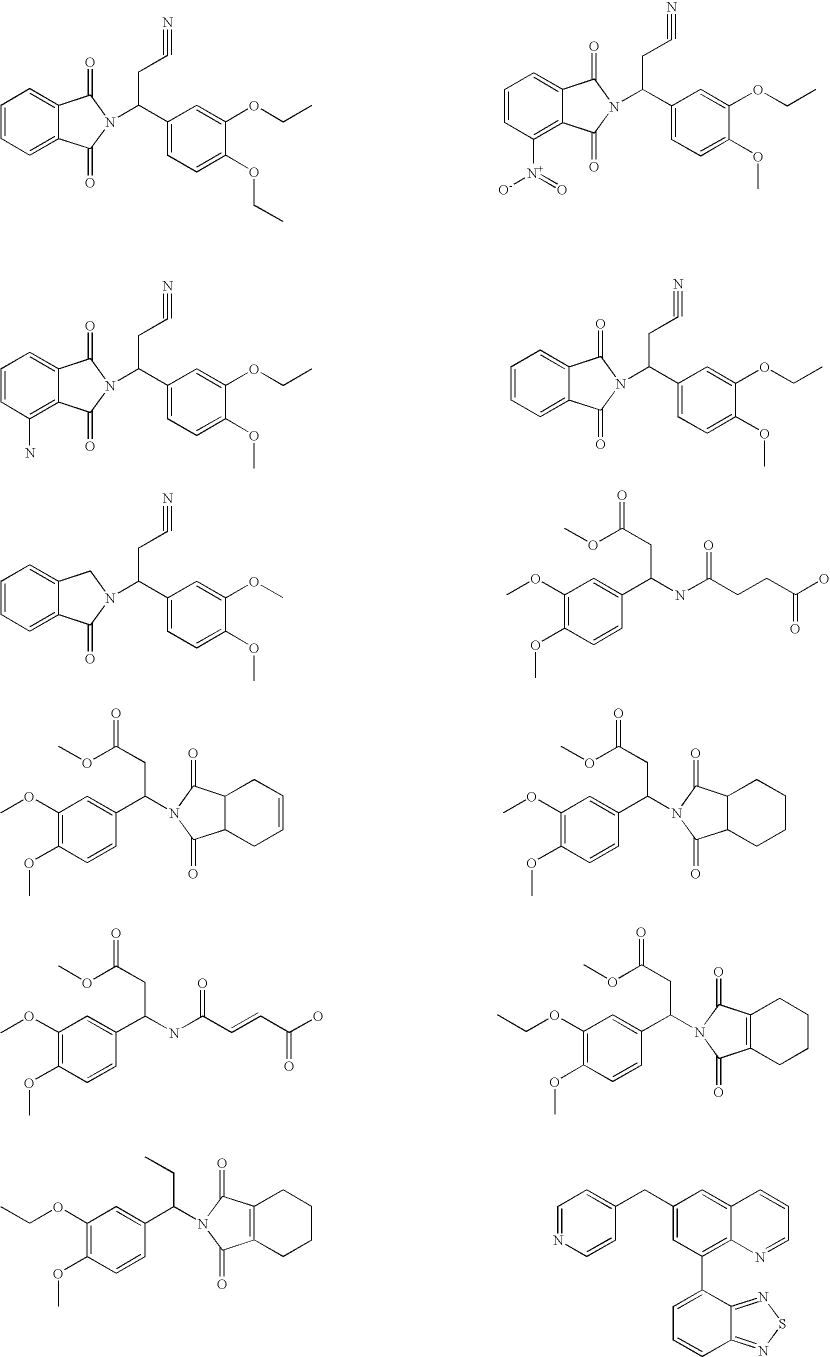

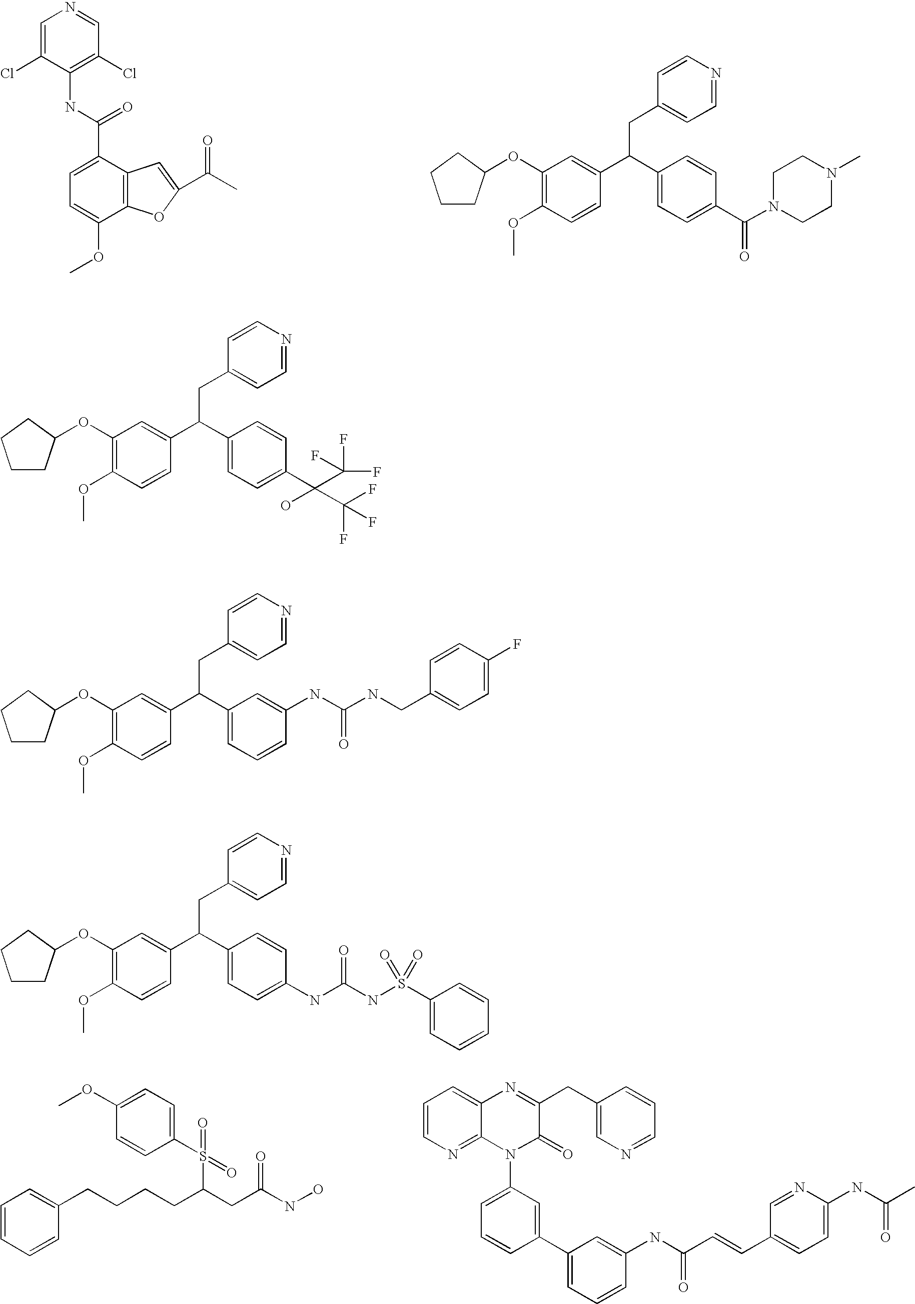

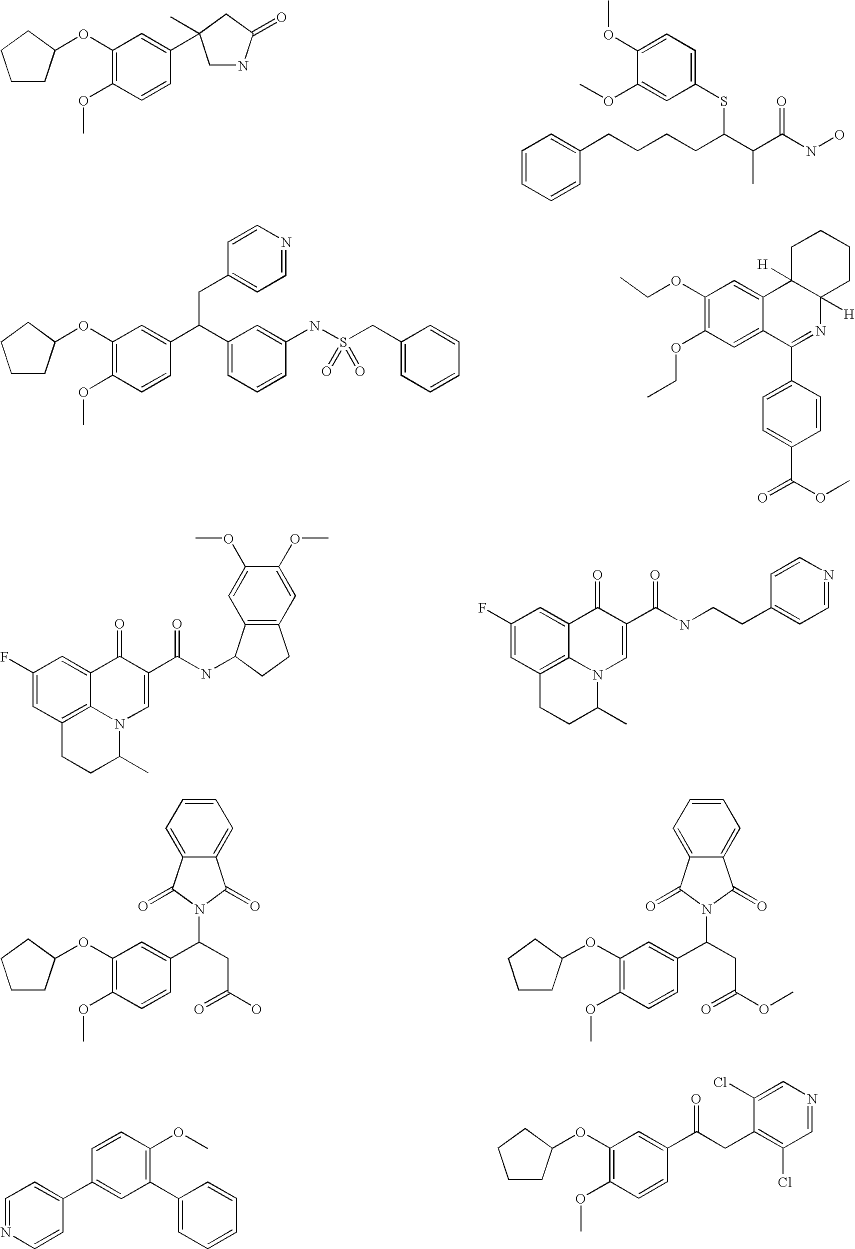

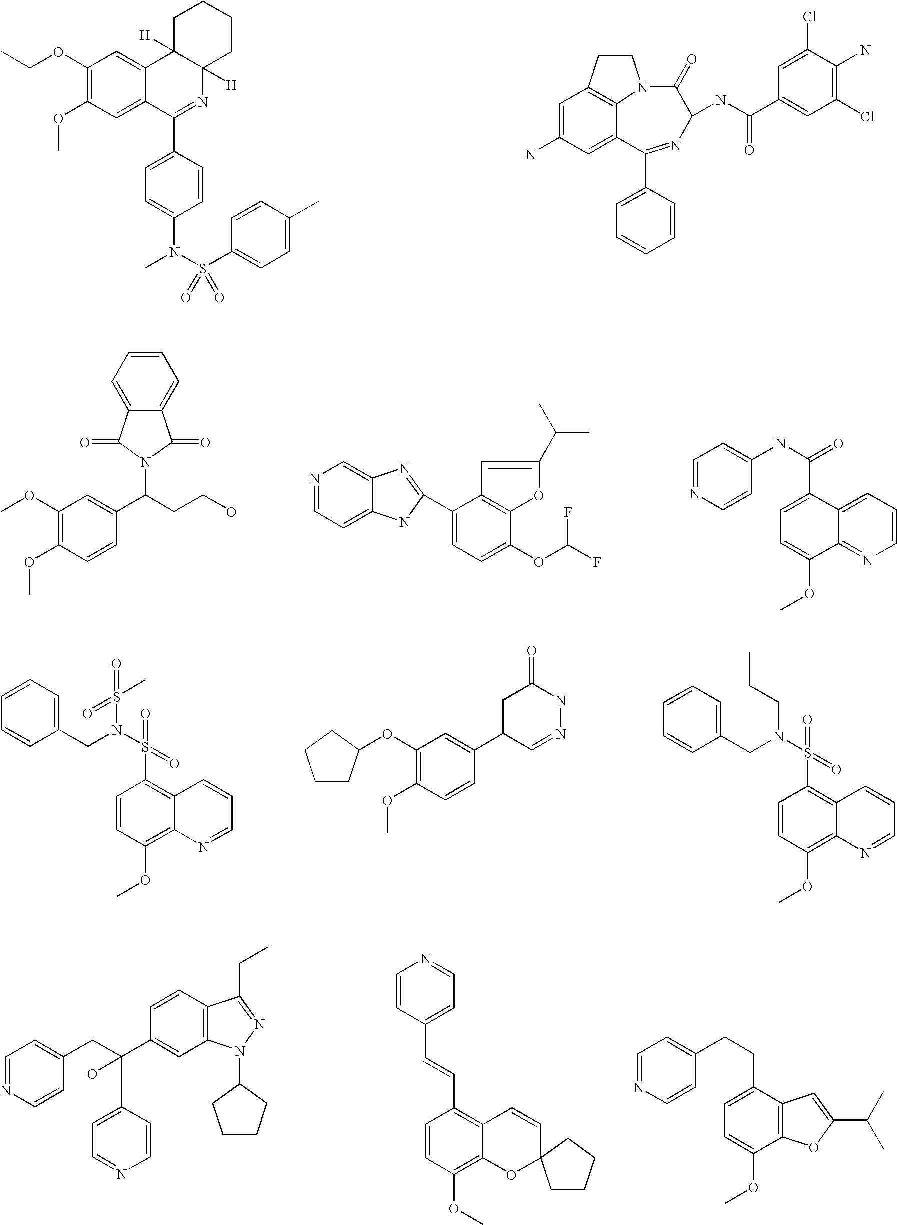

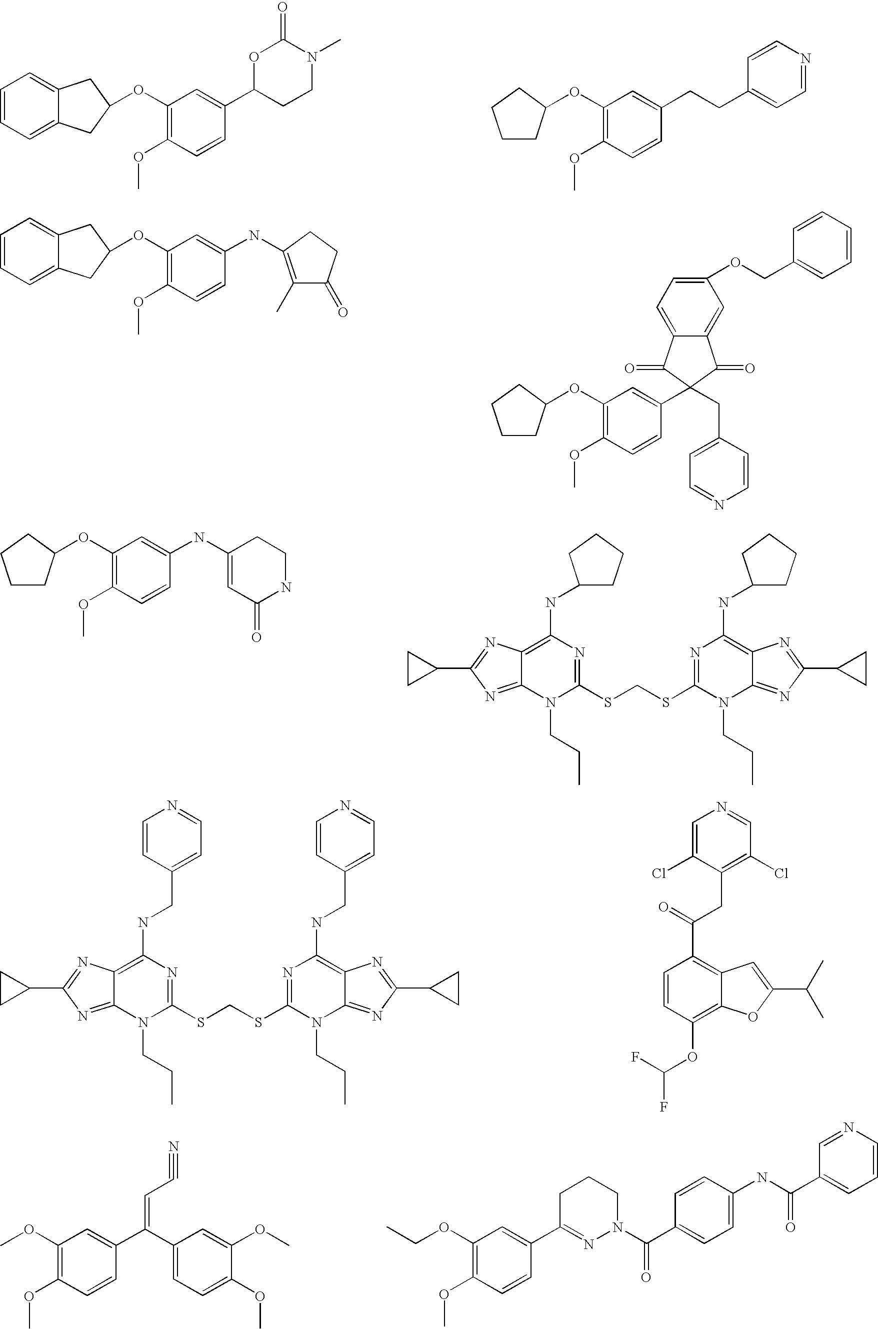

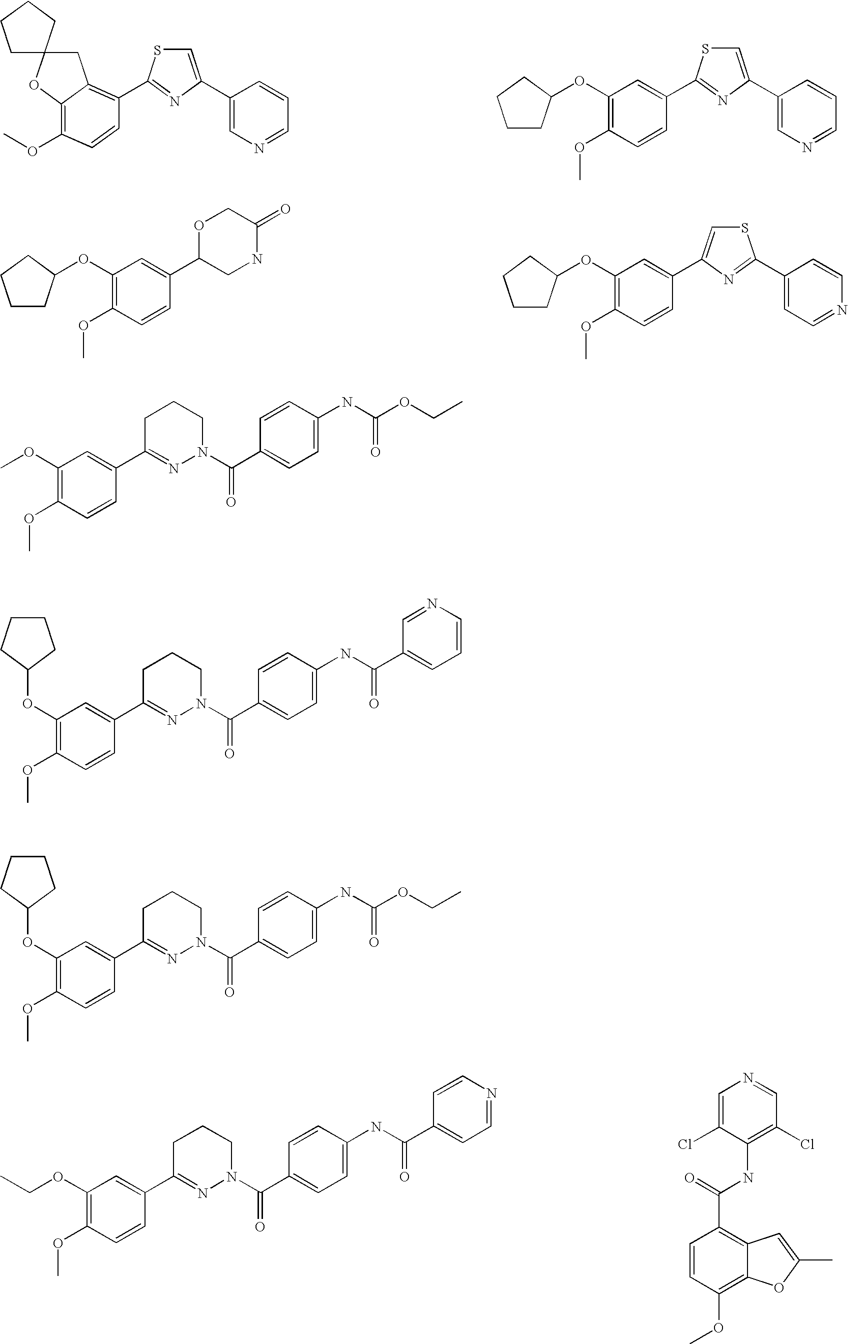

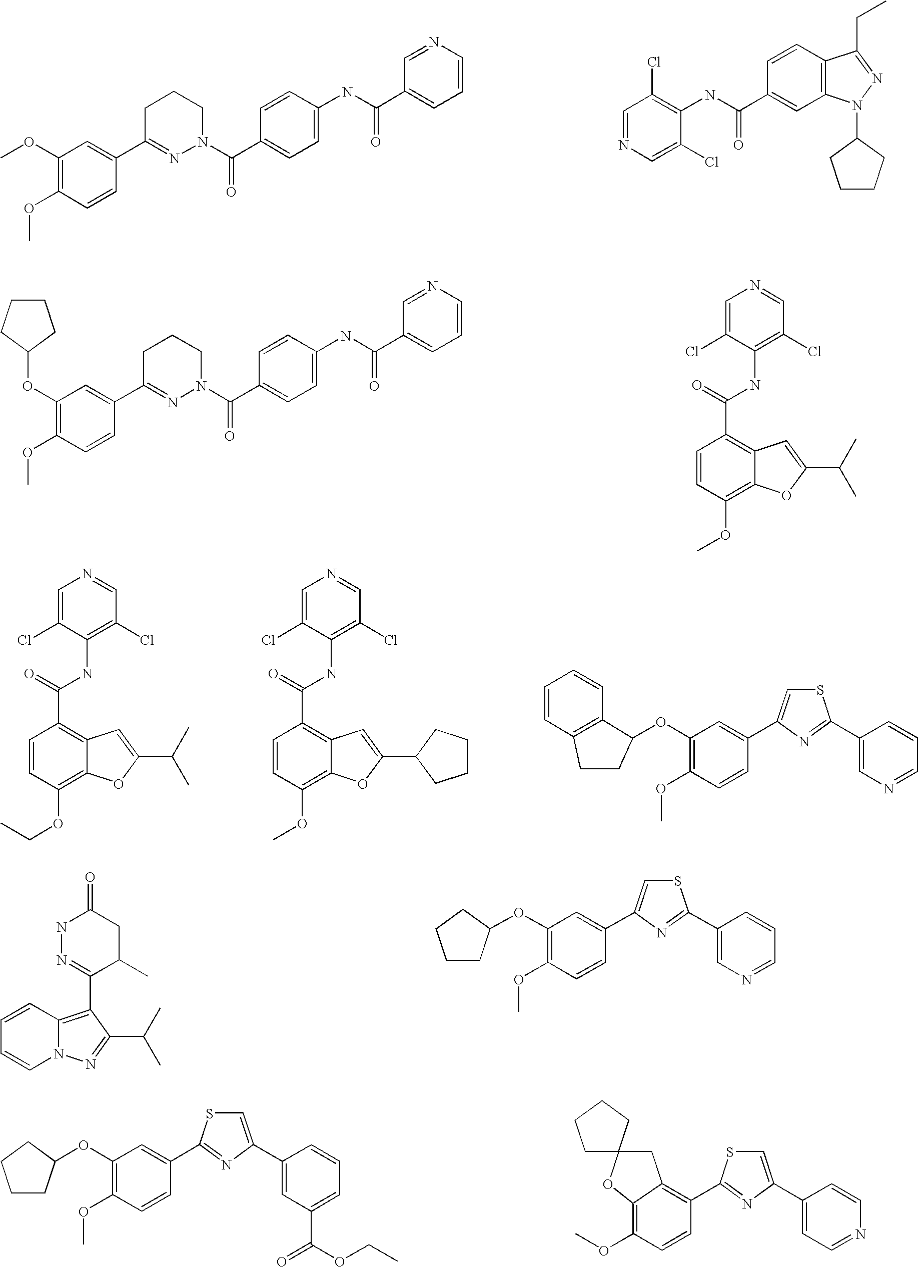

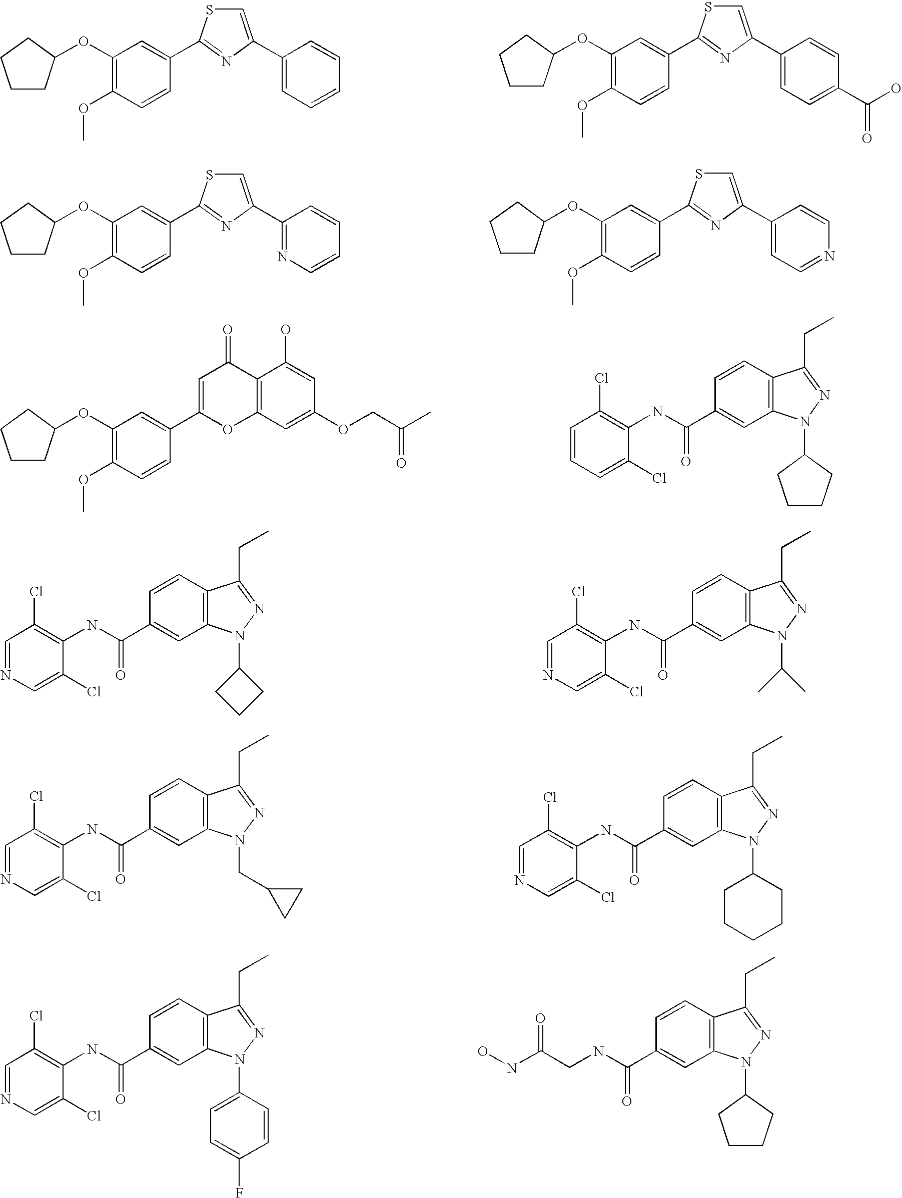

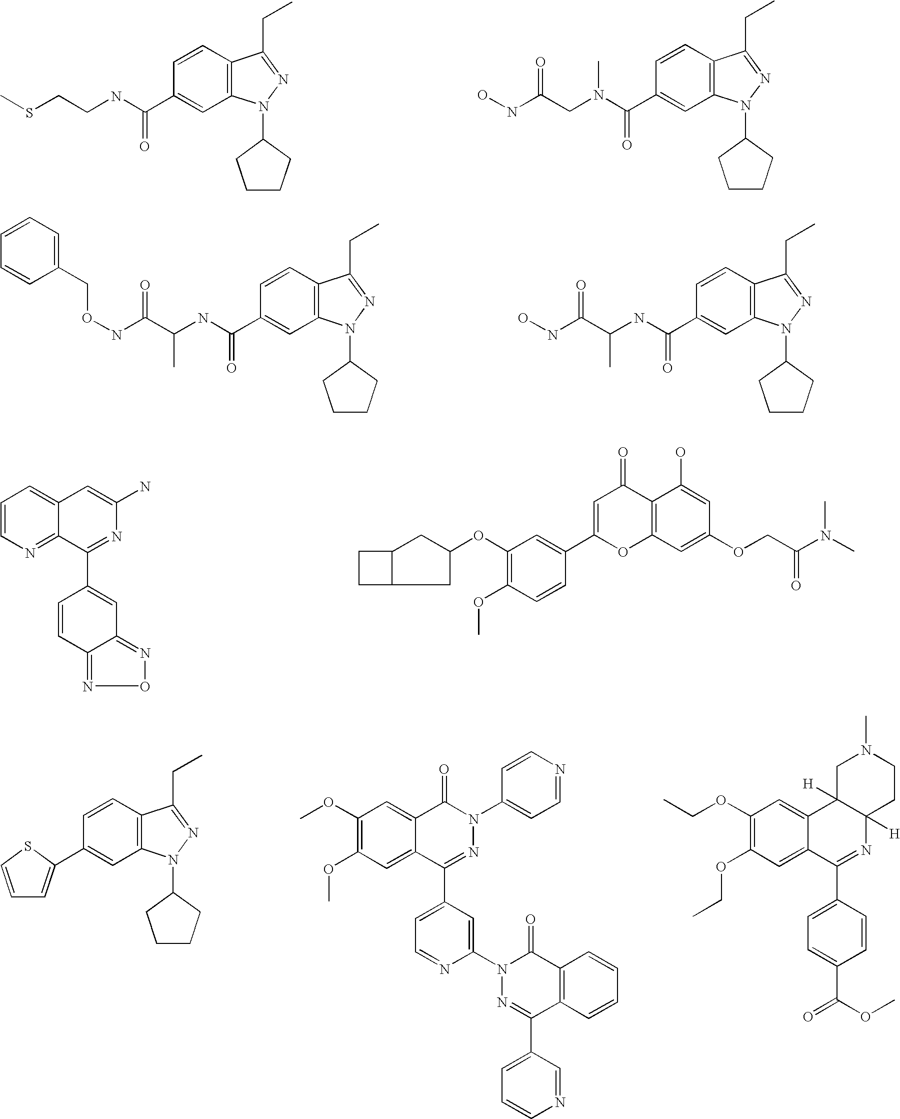

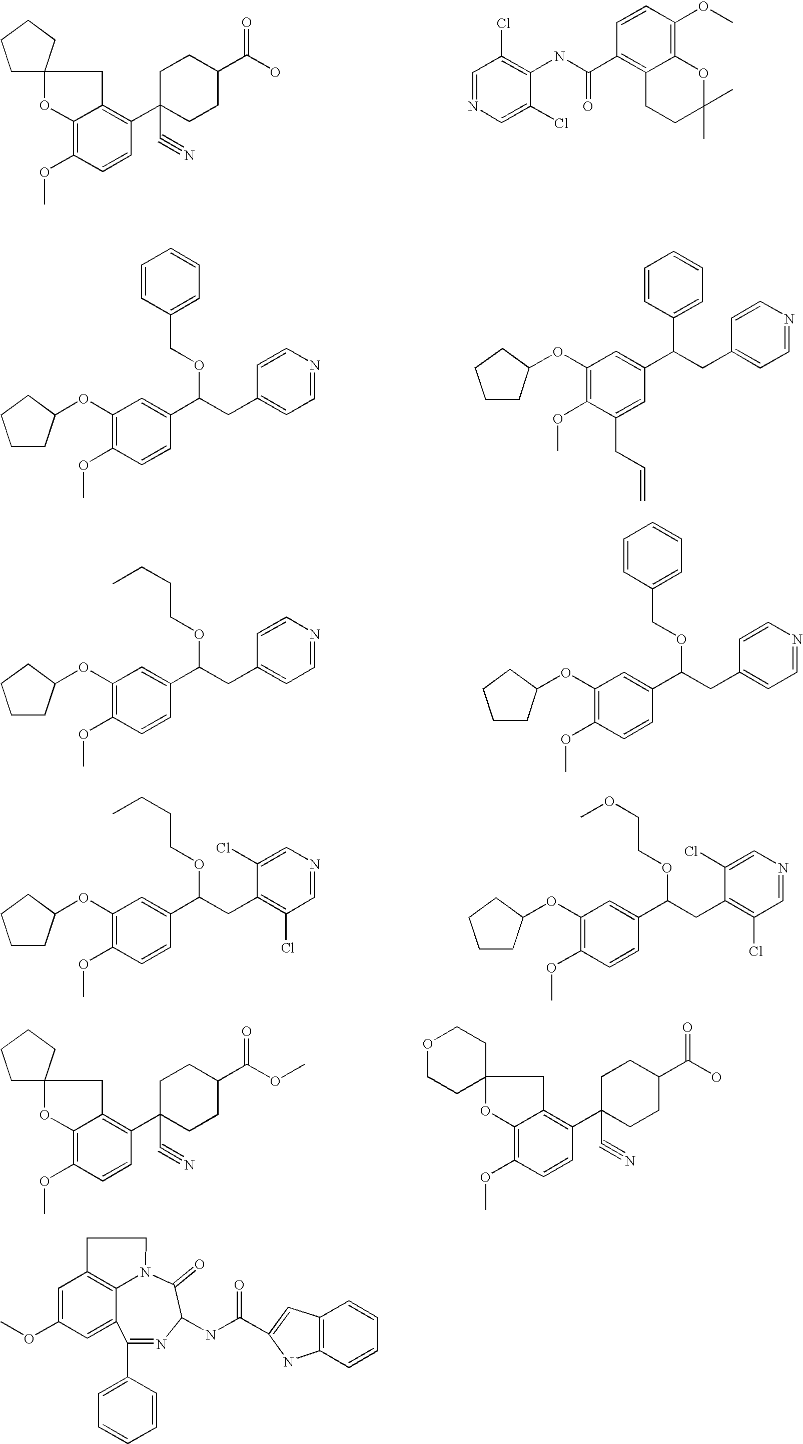

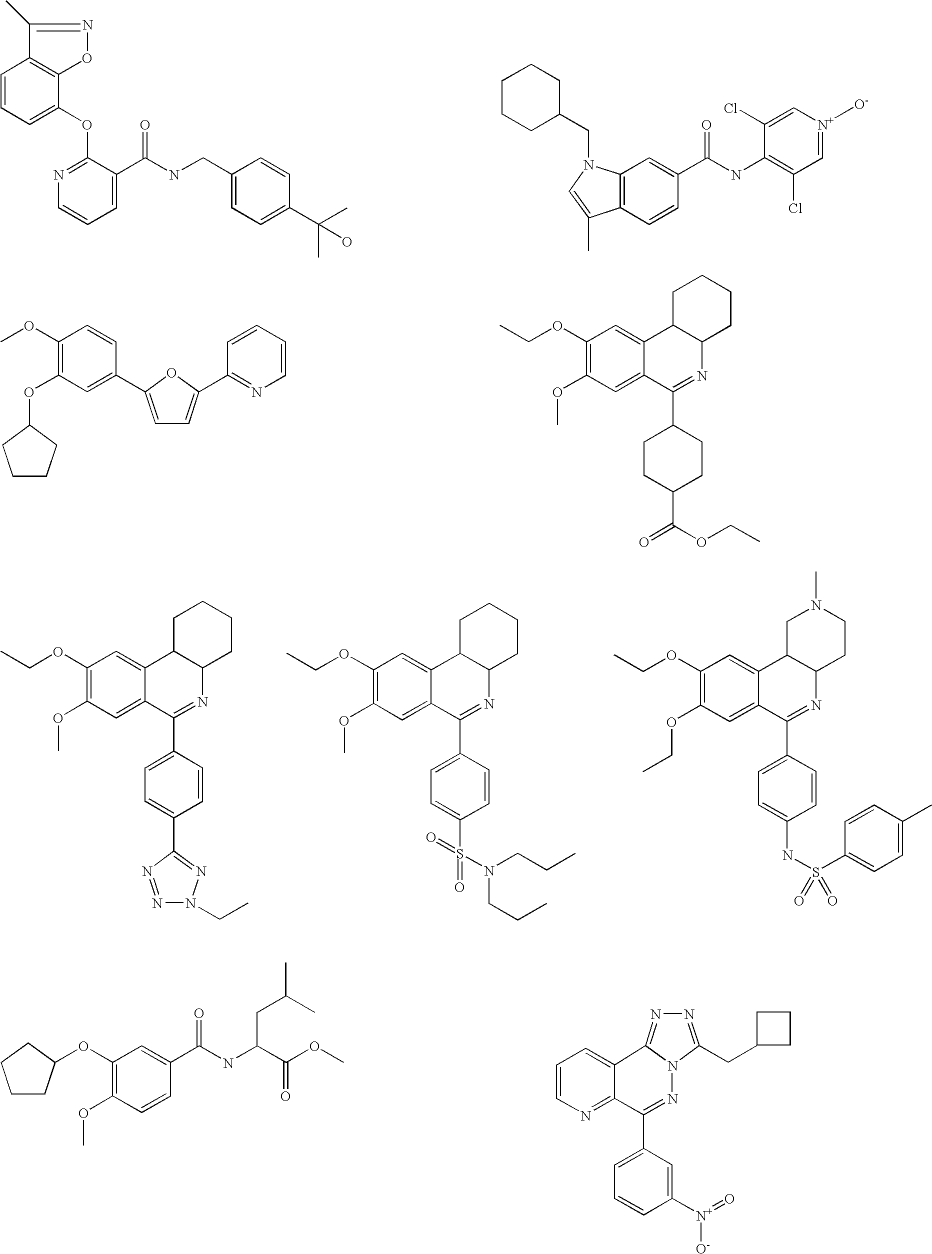

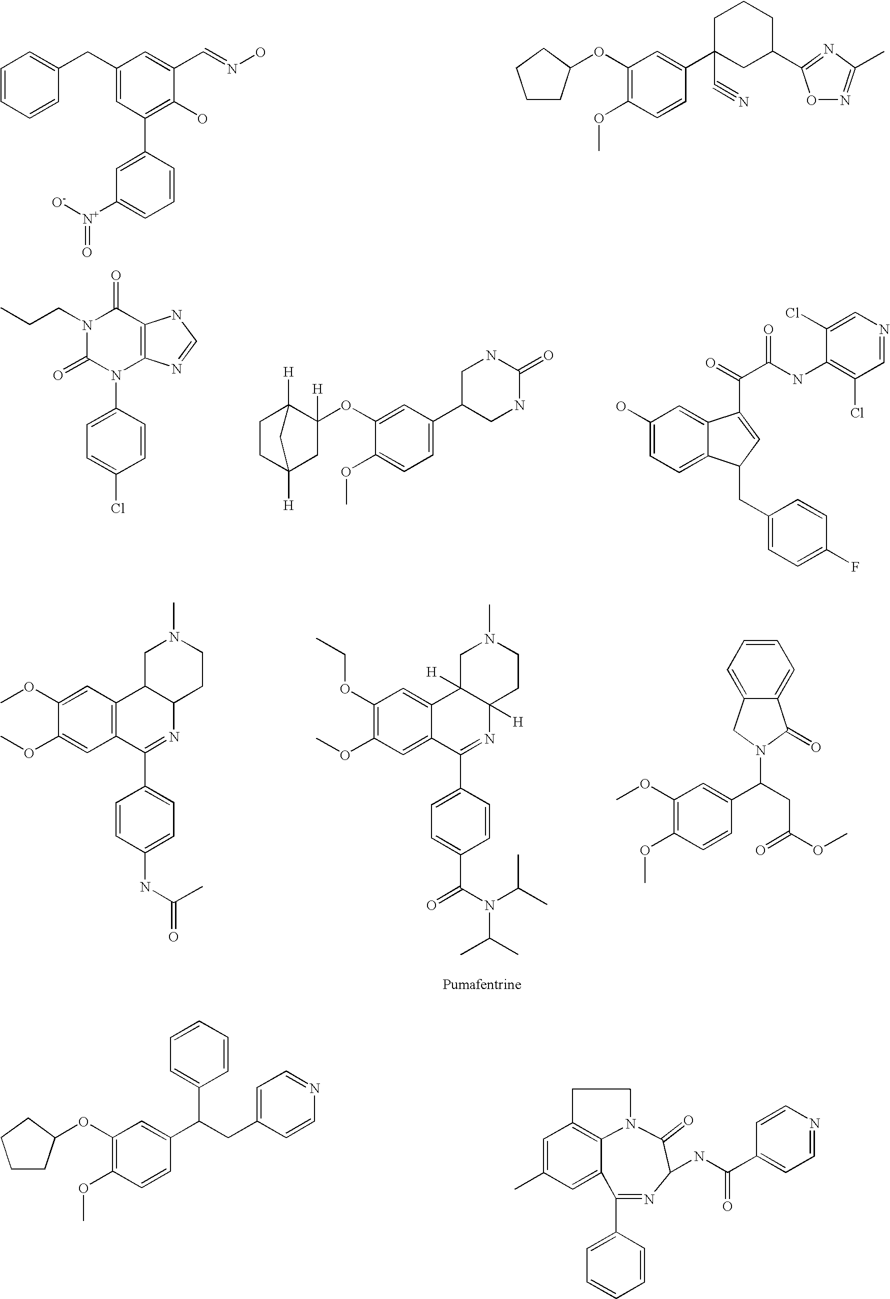

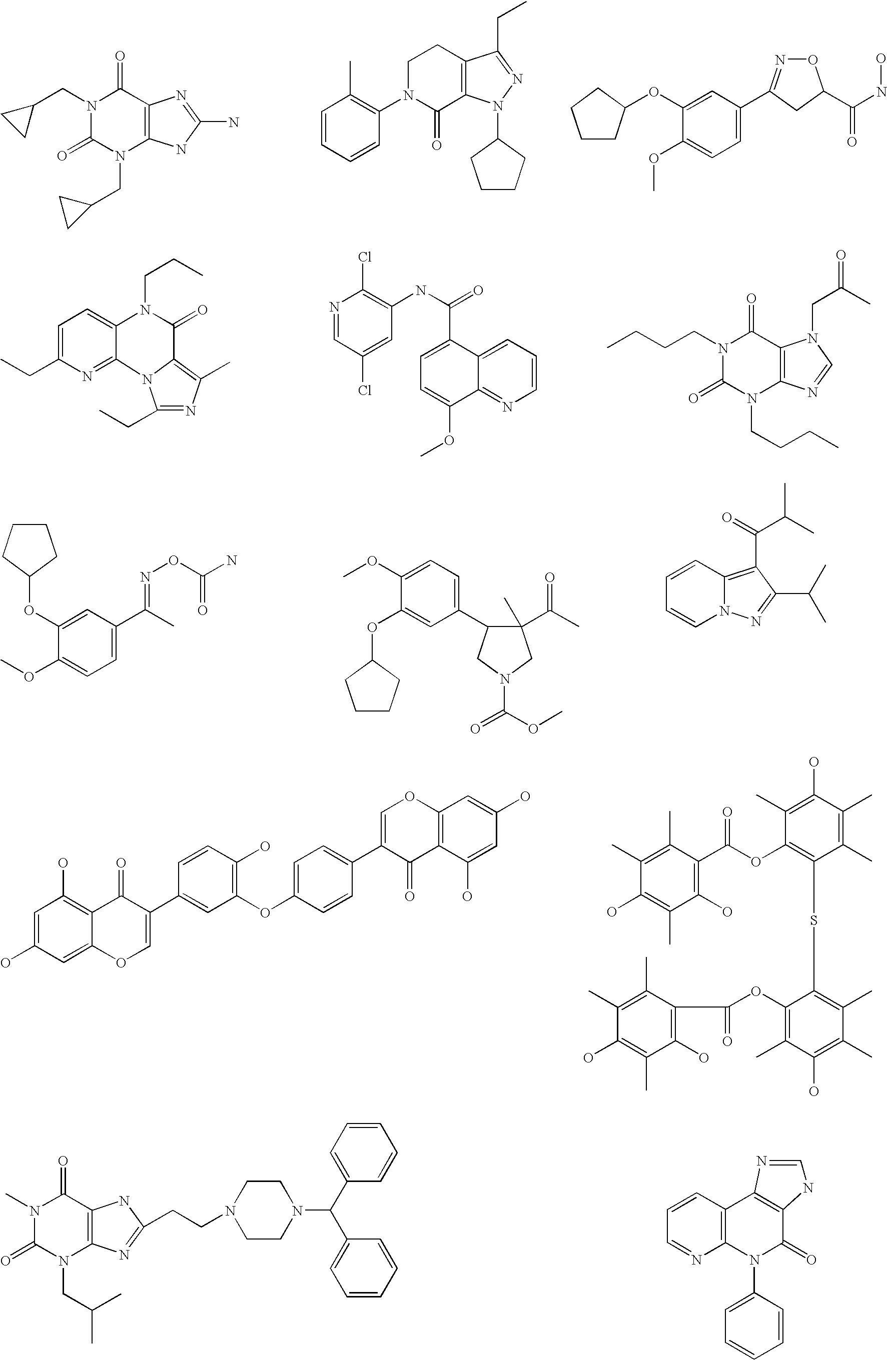

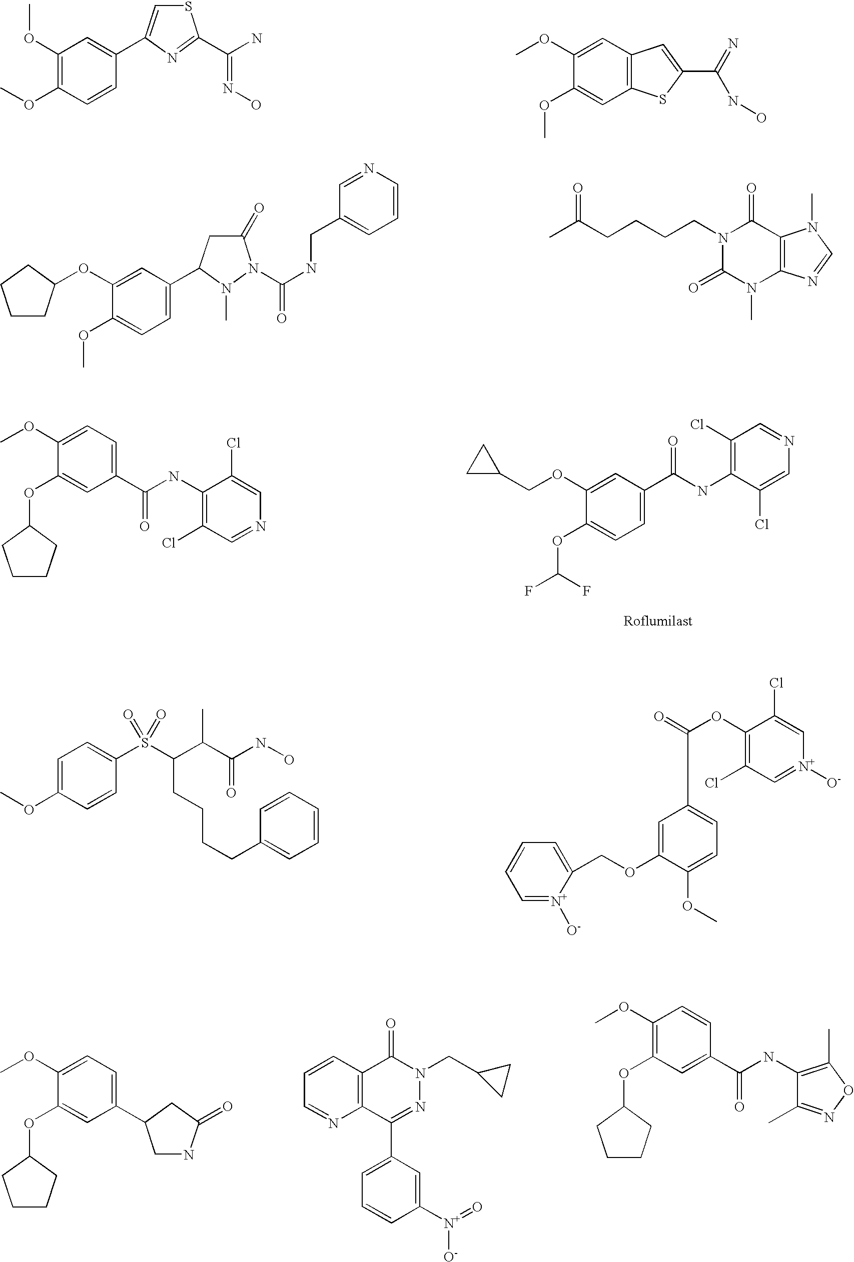

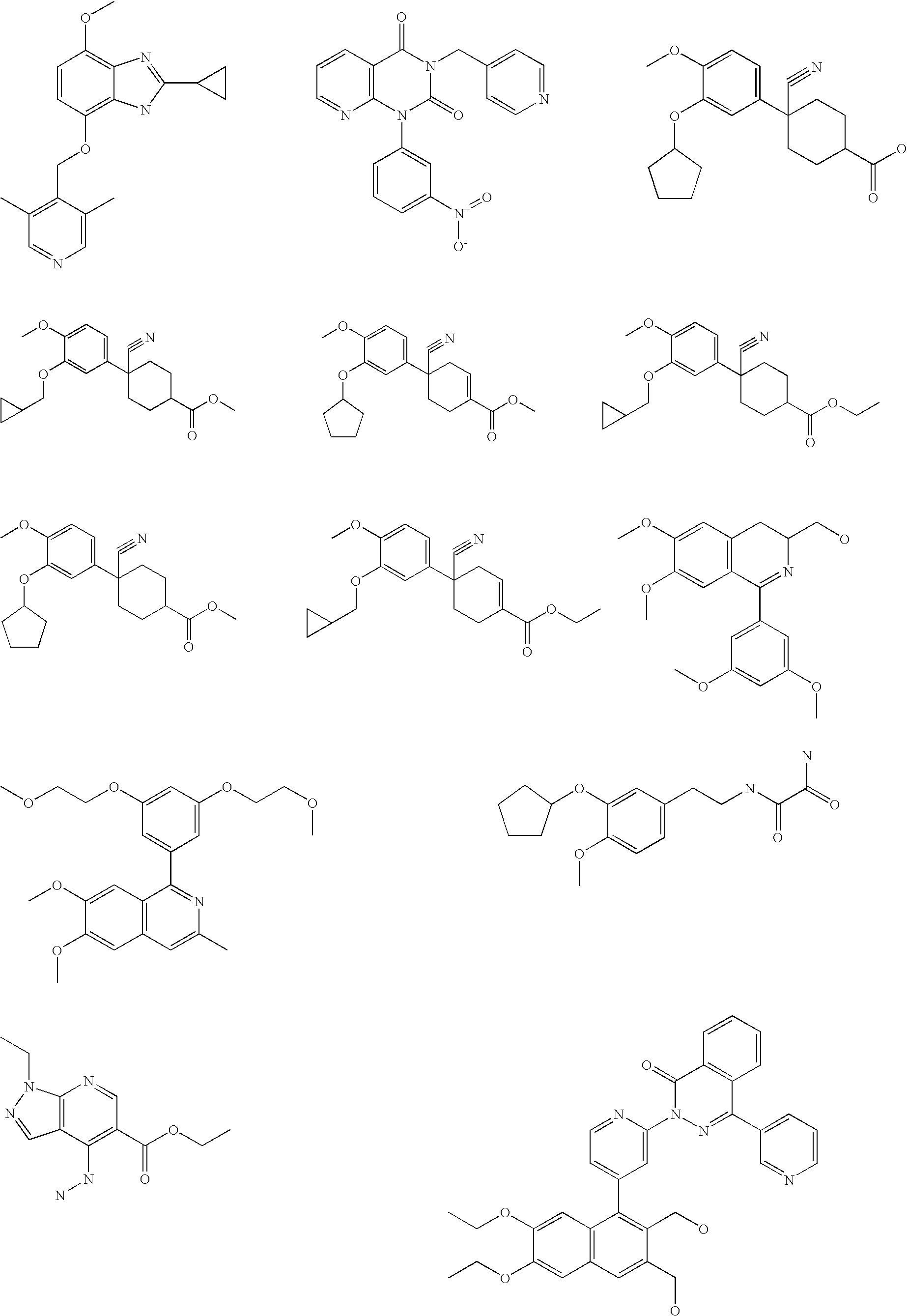

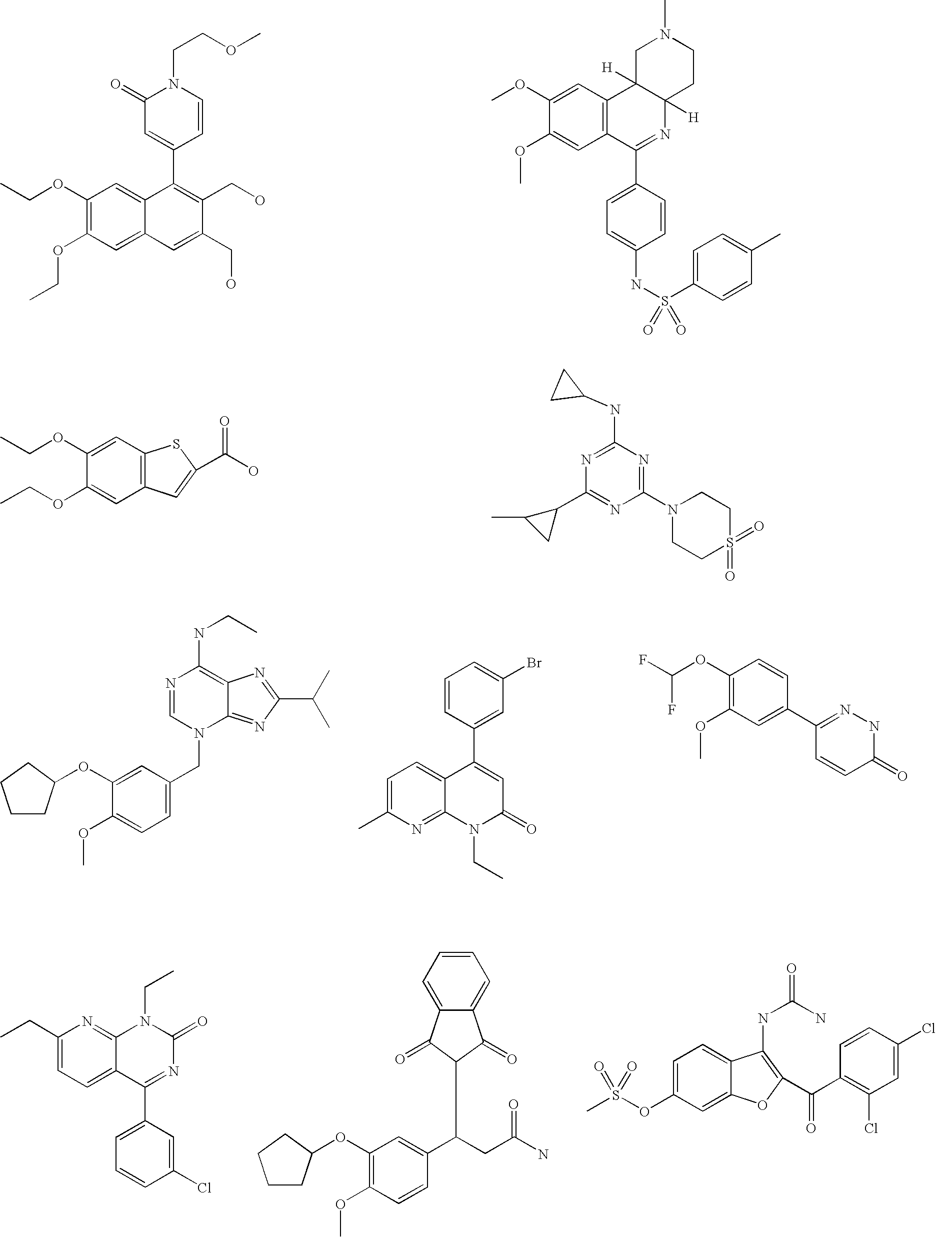

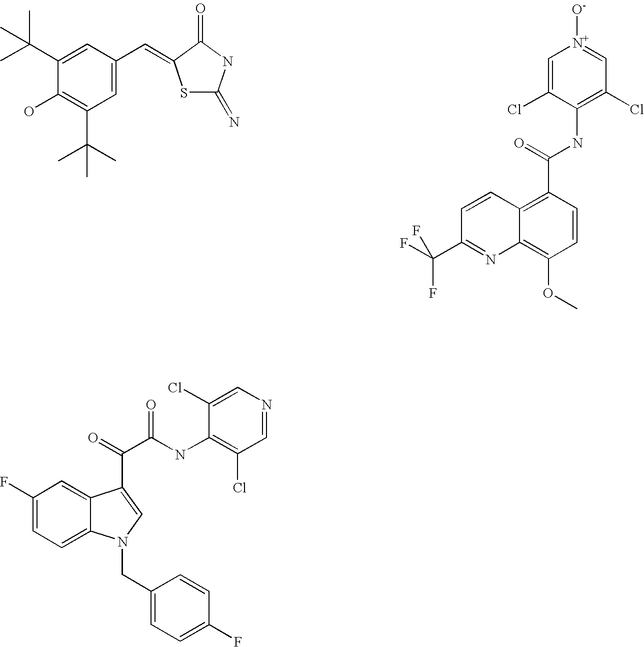

In the above formulae there is given neither any stereochemical information nor are hydrogen atoms indicated [—O is accordingly —OH, >N is >NH and —N is NH₂. Methyl groups, e.g. on the oxygen atoms, are indicated by lines].

Those PDE4 or PDE3/4 inhibitors are to be emphasized which are named expressis verbis as an example and/or claimed generically in the patent applications or patents EP 0163965, EP 0389282, EP 0393500, EP 0435811, EP 0482302, EP 0499216, EP 0506194, EP 0510562, EP 0528922, EP 0553174, EP 0731099, WO 9319749, WO 9500516, WO 9501338, WO 9600218, WO 9603399, WO 9611690, WO 9636625, WO 9636626, WO 9723457, WO 9728131, WO 9735854, WO 9740032, WO 9743288, WO 9809946, WO 9807715, WO 9808841, WO 9821207, WO 9821208, WO 9821209, WO 9822453, WO 9831674, WO 9840382, WO 9855481, WO 9905111, WO 9905112, WO 9905113, WO 9931071, WO 9931090, WO 9947505, WO 9957115, WO 9957118, WO 9964414, WO 0001695, WO 0012501, WO 0042017, WO 0042018, WO 0042019, WO 0042020, WO 0042034, WO 0119818, WO 0130766, WO 0130777, WO 0151470 WO 0206239, WO 0206270, WO 0205616 and WO 0206238 and the compounds with the following research codes: CDC-998, SH-636, D-4396, SCH-351591, IC-485, CC-1088 and KW-4490. Substances having good oral availability are preferred here.

Preferred PDE4 or PDE3/4 inhibitors are the compounds with the research codes CDC-998, SH-636, D-4396, IC-485, CC-1088 and 3,5-dichloro-4-[8-methoxy-2-(trifluoromethyl)quinoline-5-ylcarboxamido]pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetra-hydropyrrolo[3,2,1-jk][1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INNAROFYLLINE], 3-[3-(Cyclopentyloxy)-4-methoxybenzylamino]-1H-pyrazole-4-methanol, (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], N-(3,5-dichloro-4-pyridinyl-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN:CIPAM-FYLLINE], Tetrahydro-5-[4-methoxy-3-[(1S,2S,4R)-2-norbornyloxy]phenyl]-2(1H)-pyrimidone [INN: ATIZORAM], β-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST] and 3-Cyclopropylmethoxy-difluoromethoxy-N-(3,5-dichloropyrid-4-yl)-benzamide [INN: ROFLUMILAST].

Particularly preferred PDE4 or PDE3/4 inhibitors are 3-Cyclopropylmethoxy-4-difluoromethoxy-N-(3,5-dichloropyrid-4-yl)-benzamide [INN: ROFLUMILAST] and (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE].

As possible leukotriene receptor antagonists within the meaning of the present invention may be mentioned, by way of example, 3(S)-[2-(carboxyethyl)thio]-3-[2-(8-phenyloctyl)phenyl]-proplonic acid [Research Code: SKF-S-106203], N-(ethoxycarbonyl)-4-[3-[4-[1-(4-hydroxyphenyl)-1-methylethyl]phenoxymethyl]benzyloxy]benzenecarboximidamide [Research Code: BIIL-284], 5-[2-(2-carboxyethyl)-3-[6-(4-methoxyphenyl)-5(E) hexenyloxy]phenoxy]pentanoic acid [Research Code: ONO-4057], (2S,5S)-trans-2-(4-fluorophenoxymethyl)-5-(4-N-hydroxyureidyl-1-butynyl)-tetrahydrofuran [Research Code: CMI-977], 4-[6-acetyl-3-[3-(4-acetyl-3-hydroxy-2-propylphenylthio)-propoxy]-2-propylphenoxy]butric acid [Research Code: KCA-757], (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanillic acid [INN: ACITAZANOLAST], N-hydroxy-N-[1-(benzothlophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5-yl)-4H-1-benzopyran-4-one [INN: PRANLUKAST] and 2-[1-[1(R)-[3-[2(E)-(7-chloroquinolin-2-yl)vinyl]phenyl]-3-[2-(1-hydroxy-1-methylethyl)phenyl]propylsulfanylmethyl]-cyclopropyl]acetic acid [INN: MONTELUKAST].

Preferred leukotriene receptor antagonists are N-hydroxy-N-[1-(benzothiophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5yl)-4H-1-benzopyran-4-one [INN: PRANLUKAST] and 2-[1-[1(R)-[3-[2(E)-(7-chloroquinolin-2-yl)vinyl]phenyl]-3-[2-(1-hydroxy-1-methylethyl)phenyl]propylsulfanylmethyl]cyclopropyl]acetic acid [INN: MONTELUKAST].

Particularly preferred is 2-[1-[1(R)-[3-[2(E)-(7-chloroquinolin-2-yl)vinyl]phenyl]-3-[2-(1-hydroxy-1-methylethyl)phenyl]propylsulfanylmethyl]cyclopropyl]acetic acid [INN: MONTELUKAST].

In the context of the present invention, unless otherwise stated, a pharmaceutically acceptable derivative of an active ingredient means a pharmaceutically acceptable salt or solvate (e. g. hydrate), a pharmaceutically acceptable solvate of such salt, a pharmaceutically acceptable N-oxide or a pharmaceutically acceptable salt or solvate of the latter.

Suitable pharmacologically tolerable salts here are on the one hand in particular water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)-benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 1-hydroxy-2-naphthoic acid, the acids being employed in salt preparation—depending on whether it is a mono- or polybasic acid and depending on which salt is desired—in an equimolar quantitative ratio or one differing therefrom. Furthermore, the active compounds mentioned can also be present as pure enantiomers or as enantiomer mixtures in any mixing ratio.

On the other hand, salts with bases are also suitable. Examples of salts with bases which may be mentioned are alkali metal (lithium, sodium, potassium) or calcium, aluminum, magnesium, titanium, ammonium, megiumine or guanidinium salts, where here too the bases are employed in salt preparation in an equimolar quantitative ratio or one differing therefrom.

Certain of the active ingredients used in the present invention are capable of existing in stereolsomeric forms. The invention encompasses all stereolsomers of the active ingredients and mixtures thereof including racemates. Tautomers and mixtures thereof of the active ingredients are also part of the present invention.

In accordance with the present invention, there is provided in a first aspect a pharmaceutical composition comprising, in admixture, a first active ingredient which is selected from a PDE4 inhibitor, a PDE3/4 inhibitor and their pharmaceutically acceptable derivatives, and a second active ingredient which is selected from a leukotriene receptor antagonist and its pharmaceutically acceptable derivatives.

In a second aspect—which is an embodiment of the first aspect—there is provided a pharmaceutical composition comprising, in admixture, a first active ingredient which is selected from CDC-998, SH-636, D-4396, IC-485, CC-1088 and 3,5-dichloro-4-[8-methoxy-2-(trifluoromethyl)quinoline-5-yl-carboxamido]pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)-xanthine [INN:CIPAMFYLLINE], Tetrahydro-5-[4-methoxy-3-[(1S,2S,4R)-2-norbornyloxy]phenyl]-2(1H)-pyrimidone [INN: ATIZORAM], β-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST], 3-Cyclopropylmethoxy-4-difluoromethoxy-N-(3,5-dichloropyrid-4-yl)-benzamide [INN: ROFLUMILAST] and their pharmaceutically acceptable derivatives, and a second active ingredient which is selected from 3(S)-[2-(carboxyethyl)thio]-3-[2-(8-phenyloctyl)phenyl]-propionic acid [Research Code: SKF-S-106203], N-(ethoxycarbonyl)-4-[3-[4-[1-(4-hydroxyphenyl)-1-methylethyl]-phenoxymethyl]benzyloxy]benzenecarboximidamide [Research Code: BIIL-284], 5-[2-(2-carboxyethyl)-3-[6-(4-methoxyphenyl)-5(E) hexenyloxy]phenoxy]pentanoic acid [Research Code: ONO-4057], (2S,5S)-trans-2-(4-fluorophenoxymethyl)-5-(4-N-hydroxyureidyl-1-butynyl)-tetrahydrofuran [Research Code: CMI-977], 4-[6-acetyl-3-[3-(4-acetyl-3-hydroxy-2-propylphenylthio)-propoxy]-2-propylphenoxy]butyric acid [Research Code: KCA-757], (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanilic acid [INN: ACITAZANOLAST], N-hydroxy-N-[1-(benzothiophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5-yl)-4H-1-benzopyran-4-one [INN: PRANLUKAST], 2-[1-[1(R)-[3-[2(E)-(7-chloroquinolin-2-yl)vinyl]phenyl]-3-[2-(1-hydroxy-1-methylethyl)phenyl]propylsulfanylmethyl]cyclopropyl]acetic acid [INN: MONTELUKAST] and their Pharmaceutically acceptable derivatives.

In a third aspect—which is another embodiment of the first aspect—there is provided a pharmaceutical composition comprising, in admixture, a first active ingredient which is selected from (−)-cis-9-ethoxy-8-methoxy-2-methyl-1,2,3,4,4a,10b-hexahydro-6-(4-diisopropylaminocarbonylphenyl)-benzo-[c][1,6]-naphthyridine [INN: PUMAFENTRINE], 3-Cyclopropylmethoxy-4-difluoromethoxy-N-(3,5-dichloropyrid-4-yl)-benzamide [INN: ROFLUMILAST] and their pharmaceutically acceptable derivatives, and a second active ingredient which is selected from 2-[1-[1(R)-[3-[2(E)-(7-chloroquinolin-2-yl)vinyl]phenyl]-3-[2-(1-hydroxy-1-methylethyl)phenyl]propylsulfanylmethyl]cyclopropyl]acetic acid [INN: MONTELUKAST] and its pharmaceutically acceptable derivatives.

In a forth aspect the invention provides a pharmaceutical product comprising, in combination, a preparation of a first active ingredient which is selected from a PDE4 inhibitor, a PDE3/4 inhibitor and their pharmaceutically acceptable derivatives, and a preparation of a second active ingredient which is selected from a leukotriene receptor antagonist and its pharmaceutically acceptable derivatives, for simultaneous, sequential or separate use in therapy.

In a fifth aspect—which is an embodiment of the forth aspect—the invention provides a pharmaceutical product comprising, in combination, a preparation of a first active ingredient which is selected from CDC-998, SH-636, D-4396, IC-485, CC-1088 and 3,5-dichloro-4-[8-methoxy-2-(trifluoromethyl)quinoline-5-ylcarboxamido]pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzyiamino]-1H-pyrazole-4-methanol, (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R)-2-norbomyloxy]phenyl]-2(1H)-pyrimidone [INN: ATIZORAM], β-[3-(Cyclopentyloxy)-4-methoxyphenyl]-1,3-dihydro-1,3-dioxo-2H-isolndole-2-propanamide [Research-Code: CDC-801], Methanesulfonic acid 2-(2,4-dichlorophenylcarbonyl)-3-ureldobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST], 3-Cyclopropylmethoxy-4-difluoromethoxy-N-(3,5-dichloropyrid-4-yl)-benzamide [INN: ROFLUMILAST] and their pharmaceutically acceptable derivatives, and a preparation of a second active ingredient which is selected from 3(S)-[2-(carboxyethyl)thio]-3-[2-(8-phenyloctyl)phenyl]-propionic acid [Research Code: SKF-S-106203], N-(ethoxycarbonyl)-4-[3-[4-[1-(4-hydroxyphenyl)-1-methylethyl]-phenoxymethyl]benzyloxy]benzenecarboximidamide [Research Code: BIIL-284], 5-[2-(2-carboxyethyl3-[6-(4-methoxyphenyl)-5(E)hexenyloxy]phenoxy]pentanoic acid [Research Code: ONO-4057], (2S,5S)-trans-2-(4-fluorophenoxymethyl)-5-(4-N-hydroxyureidyl-1-butynyl)-tetrahydrofuran [Research Code: CMI-977], 4-[6-acetyl-3-[3-(4-acetyl-3-hydroxy-2-propylphenylthio)-propoxy]-2-propylphenoxy]butyric acid [Research Code: KCA-757], (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanilic acid [INN: ACITAZANOLAST], N-hydroxy-N-[1-(benzothlophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5-yl)-4H-1-benzopyran-4-one [INN: PRANLUKAST], 2-[1-[1(R)-[3-[2(E)-(7-chloroquinolin-2-yl)vinyl]phenyl]-3-[2-(1-hydroxy-1-methylethyl)phenyl]propylsulfanylmethyl]cyclopropyl]acetic acid [INN: MONTELUKAST] and their pharmaceutically acceptable derivatives, for simultaneous, sequential or separate use in therapy.

In a sixth aspect—which is another embodiment of the forth aspect—the invention provides a pharmaceutical product comprising, in combination, a preparation of a first active ingredient which is selected from (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], 3-Cyclopropylmethoxy-4-difluoromethoxy-N-(3,5-dichloropyrid-4-yl)-benzamide [INN: ROFLUMILAST] and their pharmaceutically acceptable derivatives, and a preparation of a second active ingredient which is selected from 2-[1-[1(R)-[3-[2(E)-(7-chloroquinolin-2-yl)vinyl]phenyl]-3-[2-(1-hydroxy-1-methylethyl)phenyl]propylsulfanylmethyl]-cyclopropyl]acetic acid [INN: MONTELUKAST] and its pharmaceutically acceptable derivatives, for simultaneous, sequential or separate use in therapy.

In a seventh aspect, the invention provides a kit comprising a preparation of a first active ingredient which is selected from a PDE4 inhibitor, a PDE3/4 inhibitor and their pharmaceutically acceptable derivatives, a preparation of a second active ingredient which is selected from a leukotriene receptor antagonist and its pharmaceutically acceptable derivatives, and instructions for the simultaneous, sequential or separate administration of the preparations to a patient in need thereof.

In a eigth aspect—which is an embodiment of the seventh aspect—the invention provides a kit comprising a preparation of a first active ingredient which is selected from CDC-998, SH-636, D-4396, IC-485, CC-1088 and 3,5-dichloro-4-[8-methoxy-2-(trifluoromethyl)quinoline-5-ylcarboxamido]pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R)-2-norbornyloxy]phenyl]-2(1H)-pyrimidone [INN: ATIZORAM], β-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazoidin-4-one [INN: DARBUFELONE], cis-[4-Cyano-4-(3-cyclopentyloxy-4-methoxyphenyl)ocyclohexane-1-carboxylic acid [INN: CILOMILAST], 3-Cyclopropylmethoxy-4-difluoromethoxy-N-(3,5-dichloropyrid-4-yl)-benzamide [INN: ROFLUMILAST] and their pharmaceutically acceptable derivatives, a preparation of a second active ingredient which is selected from 3(S)-[2-(carboxyethyl)thio]-3-[2-(8-phenyloctyl)phenyl]-propionic acid [Research Code: SKF-S-106203], N-(ethoxycarbonyl)-4-[3-[4-[1-(4-hydroxyphenyl)-1-methylethyl]-phenoxymethyl]benzyloxy]benzenecarboximidamide [Research Code: BIIL-284J, 5-[2-(2-carboxyethyl)-3-[6-(4-methoxyphenyl)-5(E) hexenyloxy]phenoxy]pentanoic acid Research Code: ONO-4057], (2S,5S)-trans-2-(4-fluorophenoxymethyl)-5-(4-N-hydroxyureidyl-1-butynyl)-tetrahydrofuran [Research Code: CMI-977], 4-[6-acetyl-3-[3-(4-acetyl-3-hydroxy-2-propylphenylthio)propoxy]-2-propylphenoxy]butyric acid [Research Code: KCA-757], (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanilic acid [INN: ACITAZANOLAST], N-hydroxy-N-[1-(benzothiophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5-yl)-4H-1-benzopyran-4-one [INN: PRANLUKAS , 2-[1-[1(R)-[3-[2(E)-(7-chloroquinolin-2-yl)vinyl]phenyl]-3-[2-(1-hydroxy-1-methylethyl)phenyl]-propylsulfanylmethyl]cyclopropyl]acetic acid [INN: MONTELUKAST] and their pharmaceutically acceptable derivatives, and instructions for the simultaneous, sequential or separate administration of the preparations to a patient in need thereof.

In a ninth aspect—which is another embodiment of the seventh aspect—the invention provides a kit comprising a preparation of a first active ingredient which is selected from (−)-cis-9-ethoxy-8-methoxy-2-methyl-1,2,3,4,4a, 10b-hexahydro-6-(4-diisopropylaminocarbonylphenyl)-benzo-[c][1,6]naphthyridine [INN: PUMAFENTRINE], 3-Cyclopropylmethoxy-4-difluoromethoxy-N-(3,5-dichloropyrid-4-yl)-benzamide [INN: ROFLUMILAST] and their pharmaceutically acceptable derivatives, a preparation of a second active ingredient which is selected from 2-[1-[1(R)-[3-[2(E)-(7-chloroquinolin-2-yl)vinyl]phenyl]-3-[2-(1-hydroxy-1-methylethyl)phenyl]propylsulfanylmethyl]cyclopropyl]acetic acid [INN: MONTELUKAST] and its pharmaceutically acceptable derivatives, and instructions for the simultaneous, sequential or separate administration of the preparations to a patient in need thereof.

It has been found that the administration of active ingredients according to the invention is advantageous because it results—in comparison to the administration of a single active ingredient from the PDE4 and PDE3/4 inhibitors or the leukotriene receptor antagonists class—in a reduced early allergic response as well as in a reduced late inflammatory airway response.

The pharmaceutical composition of the present invention may be prepared by mixing the first active ingredient with the second active ingredient.

In the above-mentioned mixing process the first active ingredient and the second active ingredient can
a) in a first step be mixed as such, afterwards be processed with pharmaceutically acceptable auxiliaries and/or excipients and finally pressed to tablets or caplets or
b) in a first step separately be processed with pharmaceutically acceptable auxiliaries and/or excipients to give granules or pellets containing each only one of the two active ingredients; the pellets or granules for their part then can be mixed in an appropriate ratio and either be pressed—optionally with further pharmaceutically acceptable auxiliaries and/or excipients—to give for example, tablets or caplets, or can be filled in more or less loose form in capsules.

Therefore, in a tenth aspect of the present invention, there is provided a process for the preparation of a pharmaceutical composition which comprises mixing a first active ingredient which is selected from a PDE4 inhibitor, a PDE3/4 inhibitor and their pharmacologically acceptable derivatives, with a second active ingredient which is selected from a leukotriene receptor antagonist and its pharmacologically acceptable derivatives.

In an eleventh aspect—which is an embodiment of the tenth aspect—there is provided a process for the preparation of a pharmaceutical composition which comprises mixing a first active ingredient which is selected from CDC-998, SH-636, D-4396, IC-485, CC-1088 and 3,5dichloro-4-[8-methoxy-2-(trifluoromethyl)quinoline-5-ylcarboxamido]pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzo-diazepin-3(R)-yl]pyrridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, (−)-cis-9-ethoxy-8-methoxy-2-methyl-1,2,3,4,4a,10b-hexahydro-6-(4-diisopropylaminocarbonylphenyl)-benzo-[c][1,6]-naphthyridine [INN: PUMAFENTRINE], N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-flouro-1-(4-fluorobenzyi)-1H-indol-3-yl]-2-oxoacetamide [Research-Code: AWD-12-343], 8-Amino-1,3-bis(cyclopropylmethyl)xanthine [INN: CIPAMFYLLINE], Tetrahydro-5-[4-methoxy-3-[(1S,2S,4R)-2-norbornyloxy]phenyl]-2(1H)-pyrimidone [INN: ATIZORAM], β-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureldobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano4-(3-cyclopentyloxy-4-methoxyphenyl)cyclohexane-1-carboxylic acid [INN: CILOMILAST], 3-Cyclopropylmethoxy-4-difluoromethoxy-N-(3,5-dichloropyrid-4-yl)-benzamide [INN: ROFLUMILAST] and their pharmacologically acceptable derivatives, with a second active ingredient which is selected from 3(S)-[2-(carboxyethyl)thio]-3-[2-(8-phenyloctyl)phenyl]-propionic acid [Research Code: SKF-S-106203], N-(ethoxycarbonyl)-4-[3-[4-[1-(4-hydroxyphenyl)-1-methylethyl]-phenoxymethyl]benzyloxy]benzenecarboximidamide [Research Code: BIIL-284], 5-[2-(2-carboxyethyl)-3-[6-(4-methoxyphenyl)-5-(E)-hexenyloxy]phenoxy]pentanoic acid [Research Code: ONO-4057], (2S,5S)-trans-2-(4-fluorophenoxymethyl)-5-(4-N-hydroxyureidyl-1-butynyl)-tetrahydrofuran [Research Code: CMI-977], 4-[6-acetyl-3-[3-(4-acetyl-3-hydroxy-2-propylphenylthio)-propoxy]-2-propylphenoxy]butyric acid [Research Code: KCA-757], (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanilic acid [INN: ACITAZANOLAST], N-hydroxy-N-[1-(benzothiophen-2-yl)ethyl]urea [INN: ZILEUTON], Cyclopentyl-3-{2-methoxy-4-[(2-methylphenylsulfonyl)carbamoyl]-benzyl}-1-methylindol-5carbamat [INN: ZAFIRLUKAST], 8-[4-(4-phenylbutoxy)benzamido]-2-(tetrazol-5-yl)-4H-1-benzopyran-4-one [INN: PRANLUKAST] and 2-[1-[1(R)-[3-[2(E)-(7-chloroquinolin-2-yl)vinyl]phenyl]-3-[2-(1-hydroxy-1-methylethyl)phenyl]propylsulfanylmethyl]cyclopropyl]acetic acid [INN: MONTELUKAST] and their pharmacologically acceptable derivatives.

In an twelfth aspect—which is another embodiment of the tenth aspect—there is provided a process for the preparation of a pharmaceutical composition which comprises mixing a first active ingredient which is selected from (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], 3-Cyclopropylmethoxy-4-difluoromethoxy-N-(3,5-dichloropyrid-4-yl)-benzamide [INN: ROFLUMILAST] and their pharmacologically acceptable derivatives, with a second active ingredient which is selected from 2-[1-[1(R)-[3-[2(E)-(7-chloroquinolin-2-yl)vinyl]phenyl]-3-[2-(1-hydroxy-1-methylethyl)phenyl]propylsulfanylmethyl]cyclopropyl]acetic acid [INN: MONTELUKAST] and its pharmacologically acceptable derivatives.

The present invention further provides the use of a pharmaceutical composition, or pharmaceutical product according to the invention in the manufacture of a medicament for the prophylaxis and/or treatment of a respiratory tract disorder.

Respiratory tract disorders which may be mentioned are in particular allergen- and inflammation-induced bronchial disorders (bronchitis, obstructive bronchitis, spastic bronchitis, allergic bronchitis, allergic asthma, bronchial asthma, COPD, allergic, seasonal and perennial rhinitis), which can be treated by the combination according to the invention also in the sense of a long-term therapy (if desired with appropriate adjustment of the dosage of the individual components to the needs at the time, for example needs subject to seasonally related variations).

In a further aspect the present invention provides a method of an effective treatment of a respiratory tract disorder which can be treated with a PDE4 inhibitor, a PDE3/4 inhibitor or a leukotriene receptor antagonist comprising the separate, sequential or simultaneous administration of i) a first amount of a PDE4 or a PDE3/4 inhibitor and ii) a second amount of a leukotriene receptor antagonist, wherein the sum of the first and second amount is a therapeutically effective amount.

Said method also include a pharmaceutical product or kit containing a PDE4 or PDE3/4 inhibitor and a written description which discloses that said PDE4 or PDE3/4 inhibitor can be administered together with a leukotriene receptor antagonist for the treatment of a respiratory tract disorder which can be treated with a PDE4 inhibitor, a PDE3/4 inhibitor or a leukotriene receptor antagonist. Likewise, said method include a pharmaceutical product or kit containing a leukotriene receptor antagonist and a written description which disdoses that said leukotriene receptor antagonist can be administered together with a PDE4 or PDE3/4 inhibitor for the treatment of a respiratory tract disorder which can be treated with a PDE4 inhibitor, a PDE3/4 inhibitor or a leukotriene receptor antagonist.

The active ingredients may, and indeed will, as part of the pharmaceutical composition, the pharmaceutical product or preparation, be used in admixture with one or more pharmaceutically acceptable auxiliaries and/or excipients.

The person skilled in the art is familiar on the basis of his/her expert knowledge with which excipients or auxiliaries are suitable for the desired pharmaceutical composition, product or preparation. In addition to solvents, gel-forming agents, tablet excipients and other active compound carriers, it is possible to use, for example, antioxidants, dispersants, emulsifiers, antifoams, flavor corrigents, preservatives, solubilizers, colorants or permeation promoters and complexing agents (e.g. cyclodextrins).

Within the meaning of the present invention, “use” is preferably understood as meaning the oral administration of both active ingredients. Further methods of administration, which may be mentioned are the parenteral, intranasal, sublingual or rectal administration of the active ingredients. The active ingredients may as well be administrated by inhalation or insufflation.

The pharmaceutical compositions or preparations according to the invention are preferably in unit dosage form such as tablets, coated tablets, pills, capsules, caplets, powders, granules, emulsions, suspensions or (sterile parenteral) solutions, metered aerosol or liquid sprays, drops ampoules transdermal patches, auto-injector devices or suppositories; the active ingredient content advantageously being between 0.1 and 95% and by appropriate choice of the excipients and the auxiliaries, it being possible to achieve a pharmaceutical administration form precisely tailored to the active ingredient(s) and/or to the desired onset of action (e.g. a sustained release form or an enteric form).

For the above-mentioned therapeutic uses the dosages administered will, of course, vary with the first and second active ingredients employed, the mode of administration, the treatment desired and the disorder indicated.

However, in general, satisfactory results will be obtained when the total daily dosage of first active ingredient(s), the PDE4 respectively the PDE3/4 inhibitors, when taken oral is in the range from 1-2000 μg/kg of body weight. In the case of the particularly preferred PDE4 inhibitor ROFLUMILAST, the daily dosage is in a range from 1-20 μg/kg of body weight. The daily dosage for the particularly preferred PDE3/4 inhibitor PUMAFENTRINE is in a range from 300-1500 μg/kg of body weight.

The total daily dosage of the second active ingredient(s), the leukotriene receptor antagonists also can vary within a wide range. In the case of the particularly preferred leukotriene receptor antagonist MONTELUKAST, the daily dosage when taken oral is in a range from 50-400 μg/kg of body weight.

### PHARMACOLOGY

Objective:

To assess the additive or synergistic inhibitory effect of the selective phosphodiesterase-4 inhibitor ROFLUMILAST combined with the leukotriene receptor antagonist MONTELUKAST both orally administered 1 h before ovalbumin (OVA) challenge on the early, mainly leukotriene-mediated (SRS-A=slow reacting substance of anaphylaxis) bronchoconstriction in anaesthetized, mechanically ventilated guinea pigs.

Animals:

Male Dunkin Hartley guinea pigs; body weight 200-250 g at sensitization and 350-500 g when performing the experiments.

Drugs, Substances and Experimental Procedure:

Ovalbumin (OVA) was used as allergen for active sensitization. Therefore, 20 μg OVA together with 20 mg Al(OH)₃ suspended in 0.5 ml 0.9% NaCl-solution (=saline) were administered i.p. to each animal on 2 consecutive days. After 2-3 weeks the animals were challenged with a single i.v. dose of 0.15 mg/kg OA suspended in saline. Drugs were suspended in a 4%-methocel-solution and administered p.o. by gavage to the conscious animals (n=10 animals per dose) 1 h before OVA-challenge:
1) ROFLUMILAST (3-cyclopropylmethoxy-4-difluoromethoxy-N-(3,5-dichloropyrid-4-yl)-benzamide) at doses of 0.01, 0.1, 1.0, and 3.0 μmol/kg (0.004, 0.04, 0.4, and 1.2 mg/kg)
2) MONTELUKAST sodium salt 2-[1-[1(R)-[3-[2(E)-(7-chloroquinolin-2-yl)vinyl]phenyl]-3-[2-(1-hydroxy-1-methylethyl)phenyl]propylsulfanylmethyl]cyclopropyl]acetic acid sodium salt) at doses of 0.03, 0.10,0.17, and 0.5 μmol/kg (0.018, 0.06, 0.1, and 0.3 mg/kg)
3) ROFLUMILAST at doses of 0.1, 0.3, and 1 μmol/kg (0.04, 0.12, and 0.4 mg/kg) in combination with MONTELUKAST sodium salt 0.03 μmol/kg (0.018 mg/kg)

Controls received the drug-free methocel-solution 1 h before OVA-challenge as placebo (n=12). OVA-challenge and lung function measurements were performed in anaesthetized and mechanically ventilated animals. Urethane was used for anesthesia and was injected i.p. at a dose of 1.5 g/kg as 12.5% solution about 50 min before OVA-challenge. To abolish spontaneous breathing, animals received 1.5 mg/kg i.v of the muscle relaxant pancuronium bromide about 14 min before OVA-challenge. To enhance the SRS-A-mediated bronchoconstriction, the animals were pretreated with: indomethacin 10 mg/kg i.v. 20 min before OVA-challenge to block the cyclooxygenase pathway and to enhance the production of lipoxygenase-mediators, mainly leukotrienes. Pyrilamine 2 mg/kg i.v. 6 min before OVA-challenge to inhibit the bronchoconstrictor effect of endogenously liberated histamine. Propranolol 0.1 mg/kg i.v. 5 min before OVA-challenge to abolish endogenous adrenergic tonus. Administration volume was 1 ml/kg for all i.v. injected compounds.

Measurement of the SRS-A-mediated Bronchoconstriction:

After injection of the muscle relaxant, the animals were ventilated at 60 breaths/min, 7 ml/kg tidal volume and 40/60% inspiration/expiration ratio using a small animal Ventilator. Dynamic lung compliance and airway conductance (=1/resistance) were calculated breath-by-breath from tidal volume and pulmonary inflation pressure and from flow and pulmonary inflation pressure, respectively, using a PC-based software program for analysis, data acquisition and archiving of the respiratory and cardiovascular parameters. The SRS-A-mediated bronchoconstriction was induced by i.v. injection of OVA. This was characterized by a decrease of compliance and conductance, which started about 1 min after OVA-injection and reached a plateau at 70-90% decrease after 4-6 min. The time-effect curve for the delta-%-decrease of compliance and conductance was determined up to 12 min post challenge. Blood pressure and heart rate were monitored to control vitality.

Data Analysis:

The area under the time-delta-%-decrease curves (AUC) was determined for each animal. On basis of the AUC-values, the inhibition of the SRS-A-induced decrease of compliance and conductance was calculated in reference to placebo. After eliminating drop-outs (Grubbs-test), dose-response curves were established, principally based on log-linear regression analysis, to determine ED₅₀-values. Pharmacodynamic effect of ROFLUMILAST combined with MONTELUKAST sodium salt was measured and compared with the calculated effects according to G. Pöch [1]. Measured and calculated ED₅₀-values were compared by an unpaired t-test for statistical significant differences.

Results:

1) ROFLUMILAST inhibited decrease of airway conductance with an ED₅₀ of 0.69 mg/kg (1.73 μmol/kg) and airway compliance with an ED₅₀ of 0.58 μmol/kg (1.45 mg/kg) (FIGS. 1 and 2).
2) MONTELUKAST sodium salt inhibited decrease of airway conductance with an ED₅₀ of 0.055 mg/kg (0.090 μmol/kg) and airway compliance with an ED₅₀ of 0.053 μmol/kg (0.086 mg/kg) (FIGS. 3 and 4).
3) ROFLUMILAST combined with MONTELUKAST sodium salt 0.018 mg/kg (0.030 μmol/kg) inhibited decrease of airway conductance with an ED₅₀ of 0.07 mg/kg (0.18 μmol/kg) and airway compliance with an ED₅₀ of 0.08 μmol/kg (0.19 mg/kg) respectively. (FIGS. 5 and 6)
4) If one calculates the ED₅₀ values according to the method described by G. Pöch [1] for ROFLUMILAST combined with MONTELUKAST sodium salt 0.018 mg/kg (0.030 μmol/kg) on the basis of the values given in 1) and 2) the ED₅₀ values come out as follows: ED₅₀=0.15 mg/kg (0.38 μmol/kg) for airway conductance and ED₅₀=0.11 (0.28 μmol/kg) for airway compliance (FIGS. 5 and 6).
5) Measured ED₅₀s are not significantly different from calculated ED₅₀s.

### SUMMARY

Inhibitory effects of ROFLUMILAST and MONTELUKAST sodium salt on SRS-A-induced bronchoconstriction are additive.

### REFERENCES

G. Pöch. Quantitative Ermittlung potenzierender oder hemmender Kombinations-wirkungen gleichsinnig wirkender Pharmaka. 1981, Arzneim. Forsch./Drug Res. 31 (II), No. 7, 1135-1140

### DESCRIPTION OF THE FIGURES

FIG. 1: Inhibition by ROFLUMILAST of OVA-induced Bronchoconstriction in SRS-A GPs (Conductance)

FIG. 2: Inhibition by ROFLUMILAST of OVA-induced Bronchoconstriction in SRS-A GPs (Compliance)

FIG. 3: Inhibition by MONTELUKAST sodium salt administered p.o. −1 h of OVA-induced Bronchoconstriction in SRS-A GPs (Conductance)

FIG. 4: Inhibition by MONTELUKAST sodium salt administered p.o. −1 h of OVA-induced Bronchoconstriction in SRS-A GPs (Compliance)

FIG. 5: Inhibition by ROFLUMILAST+MONTELUKAST sodium salt 0.018 mg/kg administered p.o. −1 h of OVA-induced Bronchoconstriction in SRS-A GPs (Conductance)

FIG. 6: Inhibition by ROFLUMILAST+MONTELUKAST sodium salt 0.018 mg/kg administered p.o. −1 h of OVA-induced Bronchoconstriction in SRS-A GPs (Compliance)

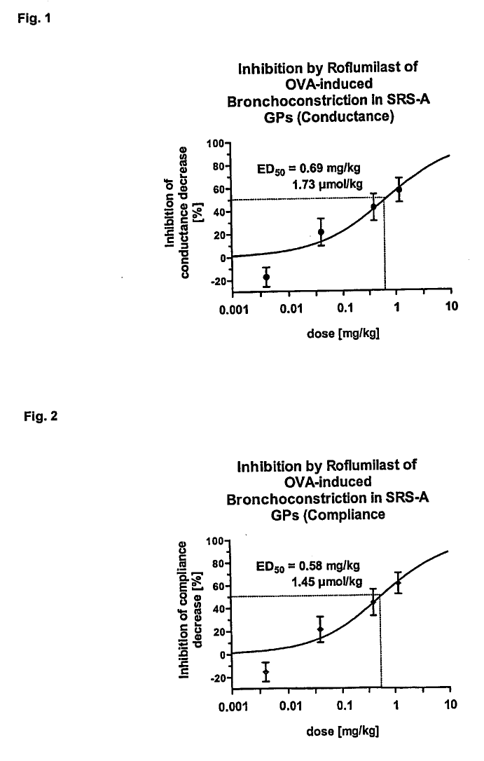

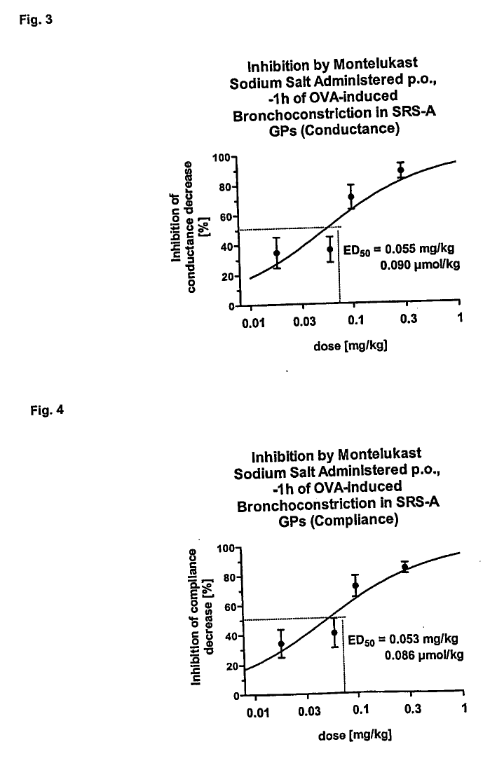

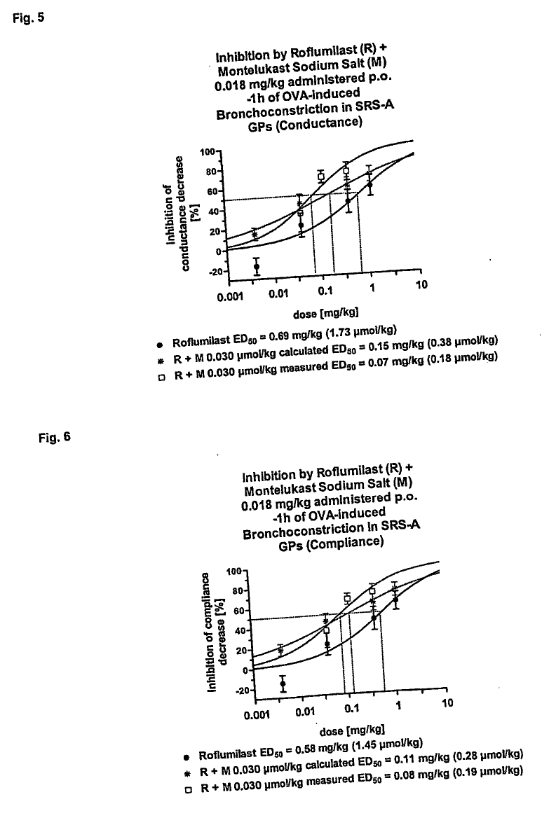

1. A pharmaceutical composition comprising, in admixture, a first active ingredient which is selected from a PDE4 inhibitor, a PDE3/4 inhibitor and their pharmaceutically acceptable derivatives, and a second active ingredient which is selected from a leukotriene receptor antagonist and its pharmaceutically acceptable derivatives.

2. A pharmaceutical composition according to claim 1, wherein the first and/or second active ingredient is in the form of a pharmaceutically acceptable salt, N-oxide, solvate of a salt or solvate of an N-oxide.

3. A pharmaceutical composition according to claim 1 which is a fixed oral combination.

4. A pharmaceutical composition according to claim 1, wherein the first active ingredient is selected from CDC-998, SH-636, D-4396, IC-485, CC-1088, 3,5-dichloro-4-[8-methoxy-2-(tri-fluoromethyl)quinoline-5-ylcarboxamido]pyridine-1-oxide [Research Code: SCH-351591], 3-[3-(cyclopentyloxy)-4-methoxybenzyl]-6-(ethylamino)-8-isopropyl-3H-purine [Research Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN: AROFYLLINE], 3-[3-(Cyclopentyloxy)-4-methoxybenzylamino]-1H-pyrazole-4-methanol, (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], N-(3,5-dichloro-4-pyridinyl)-2-[1-(4-fluorobenzyl)-5-hydroxy-1H-indol-3-yl]-2-oxoacetamide [Research Code: AWD-12-281], N-(3,5-dichloropyridin-4-yl)-2-[5-fluoro-1-(4-fluorobenzyl)-1H-indol-3-yl]-2-oxoacetamide [Research Code: AWD-12-343], 8-Amino-1,3-bis(cyclopropylmethyl)xanthine [INN: CIPAMFYLLINE], Tetrahydro-5-[4-methoxy-3-[(1S, 2S, 4R)-2-norbornyloxy]phenyl]-2-(1H)-pyrimidone ATIZORAM], β-[3-(Cyclopentyloxy)-4-methoxyphenyl]-1,3-dihydro-1,3-dioxo-2H-isoindole-2-propanamide [Research Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothioazolidin-4-one [INN: DARBUFELONE], cis-[4-Cyano-4-(3-cyclopentyloxy-4-methoxyphenyl)-cyclohexane-1-carboxylic acid [INN: CILOMAST] and 3-Cyclopropylmethoxy-4-difluoromethoxy-N-(3,5-dichloropyrid-4-yl)-benzamide [INN: ROFLUMILAST], and the second active ingredient is selected from 3(S)-[2-(carboxyethyl)thio]-3-[2-(8-phenyloctyl) phenyl]-propionic acid [Research Code: SKF-S-106203], N-(ethoxycarbonyl)-4-[3-[4-[1-[(4-hydroxyphenyl)-1-methylethyl]-phenoxy-methyl]benzyloxy]benzenecarboximidamide [Research Code: BIIL-284], 5-[2-(2-carboxyethyl)-3-[6-(4-methoxyphenyl)-5(E) hexenyloxy]phenoxy]pentanoic acid [Research Code: ONO-4057], (2S, 5S)-trans-2-(4-fluorophenoxymethyl)-5-(4-N-hydroxyureidyl-1-butynyl)-tetrahydrofuran [Research Code: CMI-977], 4-[6-acetyl-3-[3-(4-acetyl-3-hydroxy-2-propylphenylthio)-propoxy]-2-propylphenoxy]butyric acid [Research Code: KCA-757], (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanillic acid [INN: ACITAZANOLAST], N-hydroxy-N-[1-(benzothiophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5-yl)-4H-1-benzopyran-4-one [INN: PRANLUKAST] and 2-[1-[1(R)-[3-[2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST].

5. A pharmaceutical composition according to claim 1, wherein the first active ingredient is selected from (−)-cis-9-ethoxy-8-methoxy-2-methyl-1,2,3,4,4a,10b-hexahydro-6-(4-diisopropylamino-carbonylphenyl)-benzo-[c][1,6]naphthyridine [INN: PUMAFENTRINE] and 3-Cyclopropylmethoxy-4-difluoromethoxy-N-(3,5-dichloropyrid-4-yl)-benzamide [INN: ROFLUMILAST], and the second active ingredient is selected from (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanillic acid [INN: ACITAZANOLAST], N-hydroxy-N-[1-(benzothiophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5-yl)-4H-1-benzopyran-4-one [INN: PRANLUKAST] and 2-[1-[1(R)-[3-[2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST].

6. A pharmaceutical composition according to claim 5, wherein the first active ingredient is (−)-cis-9-ethoxy-8-methoxy-2-methyl-1,2,3,4,4a,10b-hexahydro-6-(4-diisopropylamino-carbonylphenyl)-benzo-[c][1,6]naphthyridine [INN: PUMAFENTRINE] or a pharmaceutically acceptable salt, N-oxide, solvate of a salt or solvate of an N-oxide thereof.

7. A pharmaceutical composition according to claim 5, wherein the first active ingredient is 3-Cyclopropylmethoxy-4-difluoromethoxy-N-(3,5-dichloropyrid-4-yl)-benzamide [INN: ROFLUMILAST] or a pharmaceutically acceptable salt, N-oxide, solvate of a salt or solvate of an N-oxide thereof.

8. A pharmaceutical composition according to claim 5, wherein the second active ingredient is 2-[1-[1(R)-[3-(2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST] or a pharmaceutically acceptable salt, N-oxide, solvate of a salt or solvate of an N-oxide thereof.

9. A method of treating or preventing a respiratory tract disorder in a patient comprising administering to a patient in need thereof a therapeutically effective amount of a pharmaceutical composition according to claim 1.

10. A process for the preparation of a pharmaceutical composition as defined in claim 1 which comprises mixing the first active ingredient with the second active ingredient.

11-18. (Canceled)

19. A kit comprising
i) a preparation of a first active ingredient which is selected from a PDE4 inhibitor, a PDE3/4 inhibitor and their pharmaceutically acceptable derivatives,
ii) a preparation of a second active ingredient which is selected from a leukotriene receptor antagonist and its pharmaceutically acceptable derivatives, and
iii) instructions for simultaneous, sequential or separate administration to a patient in need thereof.

20. A kit according to claim 19, wherein the first and/or second active ingredient is in the form of a pharmaceutically acceptable salt, N-oxide, solvate of a salt or solvate of an N-oxide.

21. A kit according to claim 19, wherein the first active ingredient is selected from (−)-cis-9-ethoxy-8-methoxy-2-methyl-1,2,3,4,4a,10b-hexahydro-6-(4-diisopropylamino-carbonylphenyl)-benzo-[c][1,6]naphthyridine [INN: PUMAFENTRINE] and 3-Cyclopropylmethoxy-4-difluoromethoxy-N-(3,5-dichloropyrid-4-yl)-benzamide [INN: ROFLUMILAST], and the second active ingredient is selected from (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanillic acid [INN: ACITAZANOLAST], N-hydroxy-N-[1-(benzothiophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5-yl)-4H-1-benzopyran-4-one [INN: PRANLUKAST] and 2-[1-[1(R)-[3-[2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST].

22. A kit according to claim 21, wherein the first active ingredient is (−)-cis-9-ethoxy-8-methoxy-2-methyl-1,2,3,4,4a,10b-hexahydro-6-(4-diisopropylamino-carbonylphenyl)-benzo-[c][1,6]naphthyridine [INN: PUMAFENTRINE] or a pharmaceutically acceptable salt, N-oxide, solvate of a salt or solvate of an N-oxide thereof.

23. A kit according to claim 21, wherein the first active ingredient is 3-Cyclopropylmethoxy-4-difluoromethoxy-N-(3,5-dichloropyrid-4-yl)-benzamide [INN: ROFLUMILAST] or a pharmaceutically acceptable salt, N-oxide, solvate of a salt or solvate of an N-oxide thereof.

24. A kit according to claim 21, wherein the second active ingredient is 2-[1-[1(R)-[3-[2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST] or a pharmaceutically acceptable salt, N-oxide, solvate of a salt or solvate of an N-oxide thereof.

25. A method of treating a respiratory tract disorder which can be treated with a PDE4 inhibitor, a PDE3/4 inhibitor or a leukotriene receptor antagonist comprising the separate, sequential or simultaneous administration of
i) a first amount of a PDE4 or a PDE3/4 inhibitor and
ii) a second amount of a leukotriene receptor antangonist,
wherein the sum of the first and second amount is a therapeutically effective amount.

26. A method of treating a respiratory tract disorder which can be treated with a PDE3/4 inhibitor or a leukotriene receptor antagonist comprising the separate, sequential or simultaneous administration of
i) a first amount of a PDE3/4 inhibitor and
ii) a second amount of a leukotriene receptor antangonist,
wherein the sum of the first and second amount is a therapeutically effective amount.

27. A pharmaceutical composition according to claim 2 which is a fixed oral combination.

28. A pharmaceutical composition according to claim 2, wherein the first active ingredient is selected from CDC-998, SH-636, D-4396, IC-485, CC-1088, 3,5-dichloro-4-[8-methoxy-2-(tri-fluoromethyl)quinoline-5-ylcarboxamido]pyridine-1-oxide [Research Code: SCH-351591], 3-[3-(cyclopentyloxy)-4-methoxybenzyl]-6-(ethylamino)-8-isopropyl-3H-purine [Research Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide (Research Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN: AROFYLLINE], 3-[3-(Cyclopentyloxy)-4-methoxybenzylamino]-1H-pyrazole-4-methanol, (−)-cis-9-ethoxy-8-methoxy-2-methyl-1,2,3,4,4a,10b-hexahydro-6-(4-diisopropylamino-carbonylphenyl)-benzo-[c][1,6]naphthyridine [INN: PUMAFENTRINE], N-(3,5-dichloro-4-pyridinyl)-2-[1-(4-fluorobenzyl)-5-hydroxy-1H-indol-3-yl]-2-oxoacetamide [Research Code: AWD-12-281], N-(3,5-dichloropyridin-4-yl)-2-[5-fluoro-1-(4-fluorobenzyl)-1H-indol-3-yl]-2-oxoacetamide [Research Code: AWD-12-343], 8-Amino-1,3-bis(cyclopropylmethyl)xanthine [INN: CIPAMFYLLINE], Tetrahydro-5-[4-methoxy-3-[(1S,2S,4R)-2-norbornyloxy]phenyl]-2-(1H)-pyrimidone [INN: ATIZORAM], β-[3-(Cyclopentyloxy)-4-methoxyphenyl]-1,3-dihydro-1,3-dioxo-2H-isoindole-2-propanamide [Research Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothioazolidin-4-one [INN: DARBUFELONE], cis-[4-Cyano-4-(3-cyclopentyloxy-4-methoxyphenyl)-cyclohexane-1-carboxylic acid [INN: CILOMAST] and 3-Cyclopropylmethoxy-4-difluoromethoxy-N-(3,5-dichloropyrid-4-yl)-benzamide [INN: ROFLUMILAST], and the second active ingredient is selected from 3(S)-[2-(carboxyethyl)thio]-3-[2-(8-phenyloctyl) phenyl]-propionic acid [Research Code: SKF-S-106203], N-(ethoxycarbonyl)-4-[3-[4-[1-[(4-hydroxyphenyl)-1-methylethyl]-phenoxy-methyl]benzyloxy]benzenecarbox-imidamide [Research Code: BIIL-284], 5-[2-(2-carboxyethyl)-3-[6-(4-methoxyphenyl)-5(E) hexenyloxy]phenoxy]pentanoic acid [Research Code: ONO-4057], (2S,5S)-trans-2-(4-fluorophenoxymethyl)-5-(4-N-hydroxyureidyl-1-butynyl)-tetrahydrofuran [Research Code: CMI-977], 4-[6-acetyl-3-[3-(4-acetyl-3-hydroxy-2-propylphenylthio)-propoxy]-2-propylphenoxy]butyric acid [Research Code: KCA-757], (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanillic acid [INN: ACITAZANOLAST], N-hydroxy-N-[1-(benzothiophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5-yl)-4H-1-benzopyran-4-one [INN: PRANLUKAST] and 2-[1-[1(R)-[3-[2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST].

29. A pharmaceutical composition according to claim 3, wherein the first active ingredient is selected from CDC-998, SH-636, D-4396, IC-485, CC-1088, 3,5-dichloro-4-[8-methoxy-2-(tri-fluoromethyl)quinoline-5-ylcarboxamido]pyridine-1-oxide [Research Code: SCH-351591], 3-[3-(cyclopentyloxy)-4-methoxybenzyl]-6-(ethylamino)-8-isopropyl-3H-purine [Research Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN: AROFYLLINE], 3-[3-(Cyclopentyloxy)-4-methoxybenzylamino]-1H-pyrazole-4-methanol, (−)-cis-9-ethoxy-8-methoxy-2-methyl-1,2,3,4,4a,10b-hexahydro-6-(4-diisopropylamino-carbonylphenyl)-benzo-[c][1,6]naphthyridine [INN: PUMAFENTRINE], N-(3,5-dichloro-4-pyridinyl)-2-[1-(4-fluorobenzyl)-5-hydroxy-1H-indol-3-yl]-2-oxoacetamide [Research Code: AWD-12-281], N-(3,5-dichloropyridin-4-yl)-2-[5-fluoro-1-(4-fluorobenzyl)-1H-indol-3-yl]-2-oxoacetamide [Research Code: AWD-12-343], 8-Amino-1,3-bis(cyclopropylmethyl)xanthine [INN: CIPAMFYLLINE], Tetrahydro-5-[4-methoxy-3-[(1S,2S,4R)-2-norbornyloxy]phenyl]-2-(1H)-pyrimidone [INN: ATIZORAM], β-[3-(Cyclopentyloxy)-4-methoxyphenyl]-1,3-dihydro-1,3-dioxo-2H-isoindole-2-propanamide [Research Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothioazolidin-4-one [INN: DARBUFELONE], cis-[4-Cyano-4-(3-cyclopentyloxy-4-methoxyphenyl)-cyclohexane-1-carboxylic acid [INN: CILOMAST] and 3-Cyclopropylmethoxy-4-difluoromethoxy-N-(3,5-dichloropyrid-4-yl)-benzamide [INN: ROFLUMILAST], and the second active ingredient is selected from 3(S)-[2-(carboxyethyl)thio]-3-[2-(8-phenyloctyl) phenyl]-propionic acid [Research Code: SKF-S-106203], N-(ethoxycarbonyl)-4-[3-[4-[1-[(4-hydroxyphenyl)-1-methylethyl]-phenoxy-methyl]benzyloxy]benzenecarboximidamide [Research Code: BIIL-284], 5-[2-(2-carboxyethyl)-3-[6-(4-methoxyphenyl)-5(E) hexenyloxy]phenoxy]pentanoic acid [Research Code: ONO-4057], (2S,5S)-trans-2-(4-fluorophenoxymethyl)-5-(4-N-hydroxyureidyl-1-butynyl)-tetrahydrofuran [Research Code: CMI-977], 4-[6-acetyl-3-[3-(4-acetyl-3-hydroxy-2-propylphenylthio)-propoxy]-2-propylphenoxy]butyric acid [Research Code: KCA-757], (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanillic acid [INN: ACITAZANOLAST], N-hydroxy-N-[1-(benzothiophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5-yl)-4H-1-benzopyran-4-one [INN: PRANLUKAST] and 2-[1-[1(R)-[3-[2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST].

30. A pharmaceutical composition according to claim 2, wherein the first active ingredient is selected from (−)-cis-9-ethoxy-8-methoxy-2-methyl-1,2,3,4,4a,10b-hexahydro-6-(4-diisopropylamino-carbonylphenyl)-benzo-[c][1,6]naphthyridine [INN: PUMAFENTRINE] and 3-Cyclopropylmethoxy-4-difluoromethoxy-N-(3,5-dichloropyrid-4-yl)-benzamide [INN: ROFLUMILAST], and the second active ingredient is selected from (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanillic acid [INN: ACITAZANOLAST], N-hydroxy-N-[1-(benzothiophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5-yl)-4H-1-benzopyran-4-one [INN: PRANLUKAST] and 2-[1-[1(R)-[3-[2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST].

31. A pharmaceutical composition according to claim 3, wherein the first active ingredient is selected from (−)-cis-9-ethoxy-8-methoxy-2-methyl-1,2,3,4,4a,10b-hexahydro-6-(4-diisopropylamino-carbonylphenyl)-benzo-[c][1,6]naphthyridine [INN: PUMAFENTRINE] and 3-Cyclopropylmethoxy-4-difluoromethoxy-N-(3,5-dichloropyrid-4-yl)-benzamide [INN: ROFLUMILAST], and the second active ingredient is selected from (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanillic acid [INN: ACITAZANOLAST], N-hydroxy-N-[1-(benzothiophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5-yl)-4H-1-benzopyran-4-one [INN: PRANLUKAST] and 2-[1-[1(R)-[3-[2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST].

32. A pharmaceutical composition according to claim 30, wherein the first active ingredient is (−)-cis-9-ethoxy-8-methoxy-2-methyl-1,2,3,4,4a,10b-hexahydro-6-(4-diisopropylamino-carbonylphenyl)-benzo-[c][1,6]naphthyridine [INN: PUMAFENTRINE] or a pharmaceutically acceptable salt, N-oxide, solvate of a salt or solvate of an N-oxide thereof.

33. A pharmaceutical composition according to claim 31, wherein the first active ingredient is (−)-cis-9-ethoxy-8-methoxy-2-methyl-1,2,3,4,4a,10b-hexahydro-6-(4-diisopropylamino-carbonylphenyl)-benzo-[c][1,6]naphthyridine [INN: PUMAFENTRINE] or a pharmaceutically acceptable salt, N-oxide, solvate of a salt or solvate of an N-oxide thereof.

34. A pharmaceutical composition according to claim 30, wherein the first active ingredient is 3-Cyclopropylmethoxy-4-difluoromethoxy-N-(3,5-dichloropyrid-4-yl)-benzamide [INN: ROFLUMILAST] or a pharmaceutically acceptable salt, N-oxide, solvate of a salt or solvate of an N-oxide thereof.

35. A pharmaceutical composition according to claim 31, wherein the first active ingredient is 3-Cyclopropylmethoxy-4-difluoromethoxy-N-(3,5-dichloropyrid-4-yl)-benzamide [INN: ROFLUMILAST] or a pharmaceutically acceptable salt, N-oxide, solvate of a salt or solvate of an N-oxide thereof.

36. A pharmaceutical composition according to claim 30, wherein the second active ingredient is 2-[1-[1(R)-[3-[2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST] or a pharmaceutically acceptable salt, N-oxide, solvate of a salt or solvate of an N-oxide thereof.

37. A pharmaceutical composition according to claim 31, wherein the second active ingredient is 2-[1-[1(R)-[3-[2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST] or a pharmaceutically acceptable salt, N-oxide, solvate of a salt or solvate of an N-oxide thereof.

38. (New) A kit according to claim 20, wherein the first active ingredient is selected from (−)-cis-9-ethoxy-8-methoxy-2-methyl-1,2,3,4,4a,10b-hexahydro-6-(4-diisopropylamino-carbonylphenyl)-benzo-[c][1,6]naphthyridine [INN: PUMAFENTRINE] and 3-Cyclopropylmethoxy-4-difluoromethoxy-N-(3,5-dichloropyrid-4-yl)-benzamide [INN: ROFLUMILAST], and the second active ingredient is selected from (R)-N-[3-[5-(4-fluorobenzyl)thien-2-yl]-1-methyl-2-propynyl]-N-hydroxy-urea [INN: ATRELEUTON], 3-(1H-tetrazol-5-yl)oxanillic acid [INN: ACITAZANOLAST], N-hydroxy-N-[1-(benzothiophen-2-yl)ethyl]urea [INN: ZILEUTON], Cyclopentyl-3-{2-methoxy-4-[(2-methylphenylsulfonyl)carbamoyl]benzyl}-1-methylindol-5-carbamat [INN: ZAFIRLUKAST], 8-[4-(4-phenylbutoxy)benzamido]-2-(tetrazol-5-yl)-4H-1-benzopyran-4-one [INN: PRANLUKAST] and 2-[1-[1(R)-[3-[2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST].

39. A kit according to claim 38, wherein the first active ingredient is (−)-cis-9-ethoxy-8-methoxy-2-methyl-1,2,3,4,4a,10b-hexahydro-6-(4-diisopropylamino-carbonylphenyl)-benzo-[c][1,6]naphthyridine [INN: PUMAFENTRINE] or a pharmaceutically acceptable salt, N-oxide, solvate of a salt or solvate of an N-oxide thereof.

40. A kit according to claim 38, wherein the first active ingredient is 3-Cyclopropylmethoxy-4-difluoromethoxy-N-(3,5-dichloropyrid-4-yl)-benzamide [INN: ROFLUMILAST] or a pharmaceutically acceptable salt, N-oxide, solvate of a salt or solvate of an N-oxide thereof.

41. A kit according to claim 38, wherein the second active ingredient is 2-[1-[1(R)-[3-[2(E)-(7-chloroquinolin-2-yl)vinyl]phenyl]-3-[2-(1-hydroxy-1-methylethyl)phenyl)propylsulfanylmethyl]cyclopropyl]acetic acid [INN: MONTELUKAST] or a pharmaceutically acceptable salt, N-oxide, solvate of a salt or solvate of an N-oxide thereof.
